(19) United States
(12) Patent Application Publication
(10) Pub. No.: US 2007/0243209 A1
(43) Pub. Date: Oct. 18, 2007

(54) COMPOSITIONS AND METHODS FOR PREVENTION AND TREATMENT OF FUNGAL DISEASES

(75) Inventors: Brahm H. Segal, Williamsville, NY (US); Masoud H. Manjili, Richmond, VA (US); John R. Subjeck, Wiliamsville, NY (US)

Correspondence Address: NIXON PEABODY LLP - PATENT GROUP, CLINTON SQUARE, P.O. BOX 31051, ROCHESTER, NY, 14603-1051, US

(73) Assignee: HEALTH RESEARCH, INC., Buffalo, NY (US)

(21) Appl. No.: 11/554,022

(22) Filed: Oct. 28, 2006

Related U.S. Application Data
(60) Provisional application No. 60/731,628, filed on Oct. 28, 2005.

(51) Int. Cl. A61K 39/385; A61K 31/711; A61K 48/00; A61P 37/00; C07H 21/04; C07K 4/06; C12N 5/06

(52) U.S. Cl. 424/193.1; 424/274.1; 424/275.1; 424/93.21; 435/372; 514/44; 530/403; 536/23.4

(57) ABSTRACT

The present invention relates to various pharmaceutical compositions that can be used as active or passive vaccines for the treatment or prevention of fungal disease. Methods for prevention and treatment of infectious and allergic fungal diseases in subjects using the pharmaceutical compositions of the present invention are also disclosed.

This application claims the priority benefit of U.S. Provisional Patent Application Ser. No. 60/731,628, filed Oct. 28, 2005, which is hereby incorporated by reference in its entirety.

The subject matter of this application was made with support from the United States Government under The National Institutes of Health (NIH) grants ROI-A146382 and T32-Al 07621. The U.S. Government may have certain rights.

### FIELD OF THE INVENTION

The present invention relates to pharmaceutical compositions having at least a portion of a stress protein or polypeptide complexed with a fungal immunogenic peptide, and the use thereof for the prevention and treatment of fungal diseases.

### BACKGROUND OF THE INVENTION

Aspergillus species are filamentous fungi (molds) that are ubiquitous in the environment. Aspergillus spp. are well-known to play a role in three different clinical settings in man: (i) opportunistic infections; (ii) allergic states; and (iii) toxicoses. Immunosuppression is the major factor predisposing to development of opportunistic infections (Ho et al., “Aspergillosis in Bone Marrow Transplant Recipients,” Oncol Hematol 34:55-69 (2000)). These infections may present in a wide spectrum, varying from local involvement to dissemination and, as a whole, are called aspergillosis. Among all filamentous fungi, Aspergillus is, in general, the most commonly isolated in invasive infections. The genus Aspergillus includes over 185 species. Around 20 species have so far been reported as causative agents of opportunistic infections in man. Among these, Aspergillus fumigatus is the most commonly isolated species, followed by Aspergillus flavus, and Aspergillus niger. Aspergillus clavatus, Aspergillus glaucus group, Aspergillus nidulans, Aspergillus oryzae, Aspergillus terreus, Aspergillus ustus, and Aspergillus versicolor are among the other species less commonly isolated as opportunistic pathogens.

Aspergillus infection is a major cause of morbidity and mortality in highly immunocompromised patients. The frequency of invasive aspergillosis has increased by more than 10-fold over the past 12 years (Denning D., “Introduction-The Aspergillus Fumigatus Genome Database,” The Institute for Genomic Research (TIGR), (2003)). Patients at risk based on reported disease frequency include neutropenic patients with leukemia (5-25%), allogeneic hematopoietic stem cell transplant (HSCT) recipients (4-30%), lung transplant recipients (17-26%), heart transplant recipients (2-13%), other solid organ transplant recipients (1-4%), AIDS (˜4%) and chronic granulomatous disease (25-40%) (Denning D., “Introduction-The Aspergillus fumigatus Genome Database,” The Institute for Genomic Research (TIGR), (2003)). Prolonged and persistent neutropenia is a critical risk factor for aspergillosis (Gerson et al., “Prolonged Granulocytopenia: the Major Risk Factor for Invasive Pulmonary Aspergillosis in Patients With Acute Leukemia,” Ann Intern Med 100:345-51 (1984)). In addition, invasive aspergillosis has become the leading cause of infection-related mortality in allogeneic HSCT recipients. Recent studies have reported the predominance of aspergillosis cases occurring in the post-engraftment rather than the neutropenic period in allogeneic HSCT recipients (Wald et al., “Epidemiology of Aspergillus Infections in a Large Cohort of Patients Undergoing Bone Marrow Transplantation,” J Infect Dis 175:1459-66 (1997); Baddley et al., “Invasive Mold Infections in Allogeneic Bone Marrow Transplant Recipients,” Clin Infect Dis 32:1319-24 (2001); Grow et al., “Late Onset of Invasive Aspergillus Infection in Bone Marrow Transplant Patients at a University Hospital,” Bone Marrow Transplant 29:15-9 (2002); Jantunen et al., “Incidence and Risk Factors for Invasive Fungal Infections in Allogeneic BMT Recipients,” Bone Marrow Transplant 19:801-8 (1997); McWhinney et al., “Progress in the Diagnosis and Management of Aspergillosis in Bone Marrow Transplantation: 13 years' Experience,” Clin Infect Dis 17:397-404 (1993); Yuen et al., “Stage-Specific Manifestation of Mold Infections in Bone Marrow Transplant Recipients: Risk Factors and Clinical Significance of Positive Concentrated Smears,” Clin Infect Dis 25:37-42 (1997); Marr et al., “Invasive Aspergillosis in Allogeneic Stem Cell Transplant Recipients: Changes in Epidemiology and Risk Factors,” Blood 100:4358-66 (2002)) with immunosuppressive therapy for graft-versus-host disease (GVHD) being a principal risk factor.

Experience in the clinic indicates that severe compromise of cellular immunity predisposes individuals to invasive aspergillosis. It has been reported that among HSCT recipients, receiving CD34-enriched or T-cell depleted transplants and lymphopenia were risk factors for invasive aspergillosis in the post-engraftment period (day 41 to 180 after transplant) (Patterson et al., “Invasive Aspergillosis. Disease Spectrum, Treatment Practices, and Outcomes,” I3 Aspergillus Study Group Medicine 79:250-60 (2000)). However, in this complex patient population, it is difficult to dissect the relative contribution of lymphopenia to the overall risk of invasive aspergillosis.

The spectrum of opportunistic fungal infections in patients with primary T-cell deficiencies is similar to patients with AIDS. Invasive aspergillosis has been reported in patients with idiopathic CD4+ lymphopenia (Saiki et al., “Acquired T Cell Specific Deficiency Other Than Acquired Immunodeficiency Syndrome (AIDS),” Intern Med 31:11-6 (1992); Viallard et al., “Aspergillosis of the Muscle in a Woman With Sarcoidosis and CD4+ Lymphocytopenia,” Clin Infect Dis 21:1345-6 (1995); Nakahira et al., “Primary Aspergillosis of the Larynx Associated With CD4+ T Lymphocytopenia,” J Laryngol Otol 116:304-6 (2002)). Severe combined immunodeficiency (SCID) is a syndrome of profoundly impaired cellular and humoral immunity (Buckley R., “Primary Immunodeficiency Diseases Due to Defects in Lymphocytes,” N Engl J Med 343:1313-24 (2000)). Case reports of invasive aspergillosis have been reported in SCID (Marcinkowski et al., “Fatal Aspergillosis With Brain Abscesses in a Neonate With DiGeorge Syndrome,” Pediatr Infect Dis J 19:1214-6 (2000); Yoshihara et al., “Successful Transplantation of Haploidentical CD34+ Selected Bone Marrow Cells for an Infantile Case of Severe Combined Immunodeficiency With Aspergillus Pneumonia,” Pediatr Hematol Oncol 19:439-43 (2002); Muller F., “Clinical Manifestations and Diagnosis of Invasive Aspergillosis in Immunocompromised Children,” Eur J Pediatr 161:563-74 (2002)). It is instructive that invasive filamentous fungal infections occur uncommonly in patients with primary T-cell deficiencies. The same is true of persons with AIDS in whom invasive aspergillosis is a relatively uncommon but devastating infection (Denning et al., “Pulmonary Aspergillosis in the Acquired Immunodeficiency Syndrome,” N Engl J Med 324:654-62 (1991); Denning et al., “NIAID Mycoses Study Group Multicenter Trial of Oral Itraconazole Therapy for Invasive Aspergillosis” (Erratum in Am J Med 97(5):497 (1994)) Am J Med 97:135-44 (1994) (Erratum in Am J Med 97(5):497 (1994); Denning D., “Therapeutic Outcome in Invasive Aspergillosis,” Clin Infect Dis 23:608-15 (1996); Holding K., “Aspergillosis Among People Infected With Human Immunodeficiency Virus: Incidence and Survival. Adult and Adolescent Spectrum of HIV Disease Project,” Clin Infect Dis 31:1253-7 (2000); Mylonakis et al., “Invasive Aspergillus Sinusitis in Patients With Human Immunodeficiency Virus Infection. Report of 2 Cases and Review,” Medicine (Baltimore) 76:249-55 (1997); Mylonakis et al., “Pulmonary Aspergillosis and Invasive Disease in AIDS: Review of 342 Cases,” Chest 114:251-62 (1998)). A low CD4 count, generally less than 100/μl, was present in almost all cases. Though co-existent neutropenia or use of corticosteroids occurred in about 50% of published cases, the remaining cases appear to have no other risk factors other than advanced AIDS. Thus, although innate effector cells (macrophages and neutrophils) are the principal mediators of protection against invasive aspergillosis, cellular immunity is also important in host defense against Aspergillus infection. This clinical experience, in addition to experiments in animal models, provides a rationale for developing vaccine-based strategies that augment both innate and cellular immunity against Aspergillus infection (Stevens D., “Vaccinate Against Aspergillosis! A Call to Arms of the Immune System,” Clin Infect Dis 38:1131-6 (2004); Romani L., “Immunity to Fungal Infections,” Nat Rev Immunol 4:1-23 (2004)).

Studies in vitro, in animal models, and limited patient data provide a rationale for pursuing strategies that augment innate and acquired immunity against Aspergillus infection. Interferon-γ (IFN-γ) is produced by lymphocytes (CD4+, CD8+, NK cells) as well as macrophages and perhaps neutrophils. It is induced by a number of signals, including IL-12 and IL-18, and in turn, induces hundreds of genes, including its own inducers. Exposure to various pathogens can stimulate at least two patterns of cytokine production by CD4+ T cells. Th1 cells are defined by production of IFN-γ, lymphotoxin and IL-2. Th2 cells are defined by production of IL-4, IL-5, IL-9, IL-10 and IL-13. The antimicrobial activity induced by IFN-γ encompasses intracellular and extracellular parasites, bacteria, fungi and viruses. In vitro, IFN-γ augmented human neutrophil oxidative response and killing of Aspergillus hyphae and acted additively with granulocyte-colony-stimulating factor (Roilides E., “Enhancement of Oxidative Response and Damage Caused By Human Neutrophils to Aspergillus Fumigatus Hyphae By Granulocyte Colony-Stimulating Factor and Gamma Interferon,” Infect Immun 61:1185-93 (1993)) and prevented corticosteroid-mediated suppression of neutrophil killing of hyphae (Roilides et al., “Prevention of Corticosteroid-Induced Suppression of Human Polymorphonuclear Leukocyte-Induced Damage of Aspergillus Fumigatus Hyphae By Granulocyte Colony-Stimulating Factor and Gamma Interferon,” Infect Immun 61:4870-7 (1993)). IFN-γ also enhanced killing of Aspergillus hyphae by human monocytes (Roilides et al., “Antifungal Activity of Elutriated Human Monocytes Against Aspergillus Fumigatus Hyphae: Enhancement by Granulocyte-Macrophage Colony-Stimulating Factor and Interferon-Gamma,” J Infect Dis 170:894-9 (1994)). In patients with chronic granulomatous disease, in vivo administration of IFN-γ augmented ex vivo neutrophil-mediated damage of Aspergillus hyphae (Rex et al., “In Vivo Interferon-Gamma Therapy Augments the in Vitro Ability of Chronic Granulomatous Disease Neutrophils to Damage Aspergillus Hyphae,” J Infect Dis 163:849-52 (1991)).

In mice, the importance of cell-mediated immunity against Aspergillus infection is well established (Cenci et al., “Interleukin-4 Causes Susceptibility to Invasive Pulmonary Aspergillosis Through Suppression of Protective Type I Responses,” J Infect Dis 180:1957-68 (1999); Cenci et al., “Th1 and Th2 Cytokines in Mice With Invasive Aspergillosis,” Infect Immun 65:564-70 (1997)). Immunization of immunocompetent mice with an Aspergillus crude filtrate resulted in memory responses mediated by antigen-specific, Th-1-committed CD4+ T-cells (Cenci et al., “T Cell Vaccination in Mice With Invasive Pulmonary Aspergillosis,” J Immunol 165:381-8 (2000)). Adoptive transfer of these cells conferred protection to neutropenic mice, establishing a “proof of principle” regarding adoptive transfer of CD4+ cells as an immune augmentation strategy in aspergillosis in neutropenia (Cenci et al., “T Cell Vaccination in Mice With Invasive Pulmonary Aspergillosis,” J Immunol 165:381-8 (2000)). In separate experiments, dendritic cells (DCs) pulsed with Aspergillus antigens induced the activation of CD4+ Th1 cells capable of conferring resistance to the infection (Bozza et al., “Vaccination of Mice Against Invasive Aspergillosis With Recombinant Aspergillus Proteins and CpG Oligodeoxynucleotides as Adjuvants,” Microbes Infect 4:1281-90 (2002)). Ito and Lyons (“Vaccination of Corticosteroid Immunosuppressed Mice Against Invasive Pulmonary Aspergillosis,” J Infect Dis 186:869-71 (2002)) showed that immunization with Aspergillus extract conferred protection against lethal Aspergillus challenge in corticosteroid-treated mice. Local delivery of unmethylated CpG oligodeoxynucleotides and the Asp f16 Aspergillus allergen resulted in the activation of airway DCs capable of inducing Th1 priming and resistance to the fungus (Bozza et al., “Vaccination of Mice Against Invasive Aspergillosis With Recombinant Aspergillus Proteins and CpG Oligodeoxynucleotides as Adjuvants,” Microbes Infect 4:1281-90 (2002)). In addition, human and murine DCs pulsed with live fungi or transfected with fungal RNA underwent maturation, based on increased expression of MHC II and costimulatory molecules and the production of IL-12 in response to conidia or conidial RNA (Bozza et al., “A Dendritic Cell Vaccine Against Invasive Aspergillosis in Allogeneic Hematopoietic Transplantation,” Blood 5:5 (2003)). DCs pulsed with conidia or transfected with conidial RNA activated antigen-specific, IFN-γ positive T-cells in vitro. Administration of donor DCs pulsed with conidia or conidial RNA to allogeneic bone marrow transplant recipient mice conferred protection against subsequent intratracheal challenge with Aspergillus that was superior to adoptive transfer of Aspergillus-specific T cells (Bozza et al., “A Dendritic Cell Vaccine Against Invasive Aspergillosis in Allogeneic Hematopoietic Transplantation,” Blood 5:5 (2003)). No protection was observed with adoptive transfer of hyphae-pulsed or hyphal RNA-transfected DCs, indicating that conidia are more likely to prime DC-mediated protective responses than hyphae.

Grazziutti et al. (“Aspergillus Fumigatus Conidia Induce a Th1-Type Cytokine Response,” J Infect Dis 176:1579-83 (1997)) showed that supernatant from cocultures of A. fumigatus conidia and human peripheral blood mononuclear cells had increased levels of IFN-γ, granulocyte-macrophage colony-stimulating factor, tumor necrosis factor-α, and IL-2, compared with unstimulated cells, but not IL-10 or IL-4. A. fumigatus also stimulated expression of lymphocyte activation molecules. In non-neutropenic patients with invasive aspergillosis, increases in levels of serum IL-10 (reflective of an anti-inflammatory response) were associated with poor outcomes, while low and decreasing levels of IL-10 predicted a positive outcome, suggesting that Th1/Th2 dysregulation with a switch to Th2 responses is associated with mortality in immunocompromised patients with aspergillosis (Roilides et al., “Elevated Serum Concentrations of IL-10 in Non-Neutropenic Patients With Invasive Aspergillosis,” J Infect Dis 183:518-20 (2001)). Hebart et al. (“Analysis of T-Cell Responses to Aspergillus Fumigatus Antigens in Healthy Individuals and Patients With Hematologic Malignancies,” Blood 100:4521-4528 (2002)) recently evaluated ex vivo T-cell cytokine production in response to stimulation with Aspergillus fumigatus extract in patients with invasive aspergillosis. A favorable response to antifungal therapy correlated with a higher IFN-γ/IL-10 ratio in culture supernatants. Such studies provide a rationale to develop strategies that skew cytokine responses to the type I phenotype.

Toll-like receptors (TLR) are a conserved family of receptors that recognize common protein and DNA pattern motifs present on microbial pathogens, and initiate signaling events related to cytokine production and T-cell and dendritic cell maturation. During the phagocytosis of pathogens, TLRs recognize pathogen specific motifs within the vacuole, distinguish between pathogens, and trigger an inflammatory response appropriate to defense against the specific organism (Underhill et al., “The Toll-Like Receptor 2 is Recruited to Macrophage Phagosomes and Discriminates Between Pathogens,” Nature 401:811-5 (1999), Ozinsky et al., “The Repertoire for Pattern Recognition of Pathogens by the Innate Immune System is Defined by Cooperation Between Toll-Like Receptors,” Proc Natl Acad Sci USA 97:13766-71 (2000)). Toll-like receptors (TLR) 2 and 4 are cell surface receptors that in association with CD14 regulate phagocytic inflammatory responses to a variety of microbial products. Activation via these receptors triggers signaling cascades, resulting in NF-κB activation and downstream signaling events. IFN-γ increases the surface expression of Toll-like receptors (TLR) 2 and 4 on monocytes and endothelial cells (Mita et al., “Toll-Like Receptor 2 and 4 Surface Expressions on Human Monocytes Are Modulated by Interferon-Gamma and Macrophage Colony-Stimulating Factor,” Immunol Lett 78:97-101 (2001), Bosisio et al., “Stimulation of Toll-Like Receptor 4 Expression in Human Mononuclear Phagocytes by Interferon-Gamma: a Molecular Basis for Priming and Synergism With Bacterial Lipopolysaccharide,” Blood 99:3427-31 (2002), Faure et al., “Bacterial Lipopolysaccharide and IFN-Gamma Induce Toll-Like Receptor 2 and Toll-Like Receptor 4 Expression in Human Endothelial Cells: Role of NF-Kappa B Activation,” J Immunol 166:2018-24 (2001)). Despite shared signaling pathways, TLR2 and TLR 4 may have opposing effects in inflammatory responses in certain settings. TLR 4 activation stimulates the Th1-inducing cytokine interleukin (IL) 12 p70 and the chemokine IFN-γ-inducible protein (IP)-10 (Re et al., “Toll-Like Receptor 2 (TLR2) and TLR 4 Differentially Activate Human Dendritic Cells,” J Biol Chem 276:37692-9 (2001)). In contrast, TLR2 stimulation does not induce IL-12 p70 and IFN-γ inducible protein (IP)-10, but causes the release of the IL-12 inhibitory p40 homodimer, which would be expected to stimulate Th2 development (Re et al., “Toll-Like Receptor 2 (TLR2) and TLR 4 Differentially Activate Human Dendritic Cells,” J Biol Chem 276:37692-9 (2001)). IFN-γ augments mRNA and surface expression of toll-like receptor 4 (TLR 4), and expression of the accessory component MD-2 and of the adapter protein MyD88 in human monocytes (Bosisio et al., “Stimulation of Toll-Like Receptor 4 Expression in Human Mononuclear Phagocytes by Interferon-Gamma: a Molecular Basis for Priming and Synergism With Bacterial Lipopolysaccharide,” Blood 99:3427-31 (2002)). IFN-γ-primed monocytes have increased lipopolysaccharide-mediated phosphorylation of the IL-1 receptor-associated kinase (IRAK; which is downstream of the MyD88 adapter protein), NF-κB activation, and TNF-α and IL-12 production (Bosisio et al., “Stimulation of Toll-Like Receptor 4 Expression in Human Mononuclear Phagocytes by Interferon-Gamma: a Molecular Basis for Priming and Synergism With Bacterial Lipopolysaccharide,” Blood 99:3427-31 (2002)). Indeed, increased TLR 4 expression and downstream signaling may be an important mechanism in which IFN-γ enhances pathogen recognition and macrophage activation and stimulates type 1 cytokine responses.

TLR-dependent antifungal pathways are highly conserved in nature as demonstrated by their presence in Drosophila (Tauszig-Delamasure et al., “Drosophila MyD88 is Required for the Response to Fungal and Gram-Positive Bacterial Infections,” Nat Immunol 3:91-7 (2002); Lemaitre et al., “The Dorsoventral Regulatory Gene Cassette Spatzle/Toll/Cactus Controls the Potent Antifungal Response in Drosophila Adults,” Cell 86:973-83 (1996)). TLRs recognize motifs on Candida (Netea et al., “The Role of Toll-Like Receptor (TLR) 2 and TLR 4 in the Host Defense Against Disseminated Candidiasis,” J Infect Dis 185:1483-9 (2002)) and Cryptococcus neoformans (Shoham et al., “Toll-Like Receptor 4 Mediates Intracellular Signaling Without TNF-Alpha Release in Response to Cryptococcus Neoformans Polysaccharide Capsule,” J Immunol 166:4620-6 (2001)) and regulate inflammatory responses. Aspergillus conidia, but not hyphae, stimulate macrophages to produce proinflammatory cytokines (TNF-α and IL-1) in a TLR 4-dependent fashion (Netea et al., “Aspergillus Fumigatus Evades Immune Recognition During Germination Through Loss of Toll-Like Receptor-4-Mediated Signal Transduction,” J Infect Dis 188:320-6 (2003)). In contrast, Aspergillus hyphae, but not conidia, stimulated production of the anti-inflammatory cytokine IL-10 through TLR2-dependent mechanisms (Netea et al., “Aspergillus Fumigatus Evades Immune Recognition During Germination Through Loss of Toll-Like Receptor-4-Mediated Signal Transduction,” J Infect Dis 188:320-6 (2003)). This switch from a proinflammatory to an anti-inflammatory state during germination may help the pathogen in evading host defense. Wang et al. (“Involvement of CD14 and Toll-Like Receptors in Activation of Human Monocytes by Aspergillus Fumigatus Hyphae,” Infect Immun 69:2402-6 (2001)) reported that both CD14 and TLR 4, but not TLR2, stimulate activation of human monocytes by A. fumigatus hyphae. Other investigators have shown conflicting results in which both TLRs 2 and 4 recognize Aspergillus hyphae, stimulate proinflammatory cytokines in effector cells, and stimulate neutrophil recruitment to the inflammatory site (Meier et al., “Toll-Like Receptor (TLR) 2 and TLR 4 Are Essential for Aspergillus-Induced Activation of Murine Macrophages,” Cell Microbiol 5:561-70 (2003); Mambula S., “Toll-Like Receptor (TLR) Signaling in Response to Aspergillus Fumigatus,” J Biol Chem 277:39320-6 (2002)). From the standpoint of the host, downregulation of inflammation to Aspergillus hyphae may have a teleological rationale. In nature, Aspergillus would not be a threatening pathogen in an immunocompetent host, in which case a robust type I cytokine response might produce inflammatory complications without a host defense benefit. However, in the setting of invasive aspergillosis in the highly immunocompromised, augmenting type I cellular immunity is likely to be beneficial. Similarly, a strategy that reduces Th2 type cytokine responses to Aspergillus would be expected to be protective against ABPA.

Heat shock proteins (HSPs) are a ubiquitous group of intracellular molecules that function as molecular chaperones in numerous processes such as protein folding, assembly, transport, and peptide trafficking and antigen processing (Manjili et al., “Immunotherapy of Cancer Using Heat Shock Proteins,” Front Biosci 7:d43-52 (2002); Manjili et al., “Cancer Immunotherapy: Stress Proteins and Hyperthermia,” Int J Hyperthermia 18:506-20 (2002)). They are induced by several environmental stressors, such as fever, oxidative stress, alcohol, inflammation, and heavy metals. HSP expression is also induced by conditions associated with injury and necrosis, including infection, trauma, and ischemic reperfusion injury. During such periods of physiologic stress, HSPs bind to exposed hydrophobic sites within polypeptides and mediate conformational changes, prevent misfolding of peptides, and facilitate peptide transport across membranes. Thus, different groups of HSPs have diverse regulatory functions during physiologic stress and injury. Moreover, HSPs are potent inducers of innate and antigen-specific immunity. Their role as “danger signals” that prime multiple host defense pathways are being exploited in vaccine development in cancer.

For many years, HSPs have been considered to be exclusively intracellular proteins with intracellular functions and their appearance outside of the cell to be artifacts, e.g., due to cell lysis. Recently, this view has changed. Cell damage is no longer considered an artifact, but to have essential functions in alarming the host to damaged or diseased tissues. The activation of DCs, necessary for the initiation of primary and secondary immune responses, can be induced by motifs present on pathogens (e.g., endotoxin) as well as endogenous danger signals released by tissues undergoing stress, damage or necrosis. Examples of endogenous danger signals include HSPs, nucleotides, reactive oxygen intermediates, extracellular matrix breakdown products, neuromediators and cytokines like the interferons (Gallucci et al., “Danger Signals: SOS to the Immune System,” Curr Opin Immunol 13:114-9 (2001); Matzinger P., “Tolerance, Danger, and the Extended Family,” Annu Rev Immunol 12:991-1045 (1994)). Necrotic, but not apoptotic, cell death releases HSPs, which deliver a partial maturation signal to dendritic cells and activate the NF-κB pathway (Basu et al., “Necrotic But Not Apoptotic Cell Death Releases Heat Shock Proteins, Which Deliver a Partial Maturation Signal to Dendritic Cells and Activate the NF-κB Pathway,” Int Immunol 12:1539-46 (2000)). HSPs released from cells may be a crucial signal that is able to activate the immune system to recognize “dangerous” physiological situations (Todryk et al., “Heat Shock Proteins Refine the Danger Theory,” Immunology 99:334-7 (2000)). This suggests that HSPs, which are clearly intracellular proteins in all living cells, may have developed a natural extracellular function related to the early evolution of the immune response. Importantly, this would not only include HSP-peptide complexes as previously envisioned, but also HSP complexes with cellular proteins, specifically mutated, damaged, and misfolded proteins.

The concept of HSPs as danger signals is relevant to both tumor immunology and infectious agents. HSP-peptide complexes purified from tumors or from cells infected with pathogens contain tumor or pathogen-derived peptides, respectively. HSP-chaperoned peptides enter antigen presenting cells (APCs) through specific receptors such as toll like receptors, scavenger receptors (LOX-1) and/or CD91 and are presented via MHC class I and II pathways, resulting in stimulation of CD8+ and CD4+ T-cells (Manjili et al., “Cancer Immunotherapy and Heat-Shock Proteins: Promises and Challenges,” Expert Opin Biol Ther 4:363-73 (2004); Delneste et al., “Involvement of LOX-1 in Dendritic Cell-Mediated Antigen Cross-Presentation,” Immunity 17:353-62 (2002)). HSPs also induce maturation of DCs and secretion of proinflammatory cytokines. Seen in this light, HSPs confer a “danger flag” to an antigen of interest that can lead to engagement of innate pathogen recognition receptors, DC activation, and elaboration of antigen specific immunity.

The usefulness of recombinant HSP110 and glucose regulated protein (GRP)170 as adjuvants in cancer vaccine development has been recently been demonstrated (PCT Application Publ. No. WO 01/23421; U.S. Applications Publ. Nos. 20050202035 and 20020039583, all to Subjeck et al.). Using newly developed purification protocols for HSP110 and GRP170, it was shown that vaccination with HSP110 or GRP170 purified from the mouse Meth A fibrosarcoma leads to complete protection (tumors initially grew, but then rapidly disappeared) (Wang et al., “Characterization of Heat Shock Protein 110 and Glucose-Regulated Protein 170 as Cancer Vaccines and the Effect of Fever-Range Hyperthermia on Vaccine Activity,” J Immunol 166:490-7 (2001)). In the murine Colon 26 tumor model, both tumor-derived HSP110 and GRP170 vaccines lead to significant growth inhibition as well as tumor specific cytotoxic T lymphocyte responses (Wang et al., “Hsp110 Over-Expression Increases the Immunogenicity of the Murine CT26 Colon Tumor,” Cancer Immunol Immunother 51:311-9 (2002)). Since APCs are presumed to mediate this process, the activity of mouse bone marrow-derived dendritic cells (BMDCs) as vaccines was also examined following the exposure to tumor-derived HSP110 or GRP170. Mice treated with BMDCs that were pulsed with HSP110 or GRP170 purified from tumor elicited a strong anti-tumor response, which allowed for examination of the mechanisms of uptake and processing of HSP110- and GRP170-protein antigen complexes by antigen presenting cells.

When purified from a tumor, certain heat shock proteins (including HSP110 and GRP170) can function as effective vaccines against the same tumor. However, purification of HSP from a tumor requires a sufficient surgical specimen as a source that is often lacking and only a limited number of proteins are likely to be antigenic (PCT Application Publ. No. WO 01/23421; U.S. Patent Applications Publ. Nos. 20050202035 and 20020039583, all to Subjeck et al.). Complexing HSP110 with well characterized recombinant tumor associated antigens avoids these limitations. HSP110 is a highly efficient molecular chaperone in binding to large protein substrates (Manjili et al., “Development of a Recombinant HSP 110-HER-2/Neu Vaccine Using the Chaperoning Properties of HSP110,” Cancer Res 62:1737-42 (2002)). It was demonstrated that HSP110 complexed with the intracellular domain (ICD) of Her-2/neu elicits strong antigen-specific cellular and humoral immune responses (Banerjee et al., “Immunological Characterization of Asp f2, a Major Allergen from Aspergillus Fumigatus Associated with Allergic Bronchopulmonary Aspergillosis,” Infection and Immunity 66(11):5175-5182 (1998); PCT Application Publ. No. WO 01/23421; U.S. Patent Applications Publ. Nos. 20050202035 and 20020039583, all to Subjeck et al.) This tumor vaccine was protective in mice based on significant inhibition of tumor growth after challenging with mammary tumor cells (Manjili et al., “Development of a Recombinant HSP 110-HER-2/Neu Vaccine Using the Chaperoning Properties of HSP110,” Cancer Res 62:1737-42 (2002)). Splenocytes from HSP110-ICD immunized animals elicited significant IFN-γ production upon stimulation with ICD in vitro (Manjili et al., “Development of a Recombinant HSP110-HER-2/Neu Vaccine Using the Chaperoning Properties of HSP 110,” Cancer Res 62:1737-42 (2002)). Sensitization with the HSP110-ICD complex was as effective as Complete Freund's Adjuvant (CFA) in eliciting antigen-specific IFN-γ responses in splenocytes challenged ex-vivo with ICD, as determined by ELISPOT assay. CFA is a highly potent and toxic immuno-adjuvant often used in animal studies, but not in humans. Splenocytes from mice immunized with ICD only did not show IFN-γ production upon stimulation with the ICD antigen. Vaccination with the HSP110-ICD complex induced both CD8+ and CD4+ T cell-mediated immune responses, and CD8+ T-cell activation was unaffected by CD4+ T-cell depletion (Manjili et al., “Development of a Recombinant HSP110-HER-2/Neu Vaccine Using the Chaperoning Properties of HSP110,” Cancer Res 62:1737-42 (2002)). In a separate study, sensitization with mouse HSP110 complexed to human melanoma-associated antigen gp100 was significantly more effective in controlling tumor following challenge with B16 melanoma than sensitization with CFA and gp100 (Wang et al., “Targeted Immunotherapy Using Reconstituted Chaperone Complexes of Heat Shock Protein 110 and Melanoma-Associated Antigen gp100,” Cancer Res 63(10):2553-60 (2003)). The HSP110/gp100 complex elicited potent antigen-specific IFN-γ production and cytotoxic T-cell responses.

Historically, the therapy of serious fungal infection has been dominated by monotherapy with the polyene antibiotic amphotericin B. Despite the long-standing availability of amphotericin B, a potent fungicidal agent acting principally at the level of the fungal cell membrane, the prognosis of invasive aspergillosis has been poor. In a review of 595 patients with invasive aspergillosis, a complete response occurred in only 25%, and 65% of patients treated with amphotericin B died (Patterson et al., “Invasive Aspergillosis. Disease Spectrum, Treatment Practices, and Outcomes,” I3 Aspergillus Study Group Medicine (Baltimore) 79:250-60 (2000)). Lipid formulations of amphotericin B, which allow for greater amounts of drug delivery, may provide a more favorable prognosis, based on open label or compassionate use studies employing historical controls (Ostrosky-Zeichner et al., Amphotericin B: Time for a New ‘Gold Standard’,” Clin Infect Dis 37(3):415-25 (2003); Hiemenz et al., “Lipid Formulations of Amphotericin B: Recent Progress and Future Directions,” Clin Infect Dis 22 Suppl 2:S133-44 (1996); Walsh et al., “Amphotericin B Lipid Complex for Invasive Fungal Infections: Analysis of Safety and Efficacy in 556 Cases,” Clin Infect Dis 26:1383-96 (1998); Mills et al., “Liposomal Amphotericin B in the Treatment of Fungal Infections in Neutropenic Patients: A Single-Centre Experience of 133 Episodes in 116 Patients,” Br J Haematol 86:754-60 (1994)). Voriconazole, a second generation antifungal triazole, was superior to conventional amphotericin B as initial therapy for invasive aspergillosis (Herbrecht et al., “Voriconazole Versus Amphotericin B for Primary Therapy of Invasive Aspergillosis,” N Engl J Med 347:408-15 (2002)). However, the poorest prognosis occurred in allogeneic HSCT recipients in whom only 32% of patients receiving voriconazole and 13% receiving amphotericin B had a successful outcome (Herbrecht et al., “Voriconazole Versus Amphotericin B for Primary Therapy of Invasive Aspergillosis,” N Engl J Med 347:408-15 (2002)). Thus, there is a critical unmet need to develop novel antifungal strategies, among which is immune augmentation. Clinical failures, side effects, the lack of alternatives, and the toxicity of the current anti-fungal drugs have heightened the need to produce improved prophylactic and therapeutic treatments for diseases caused by fungal agents.

The present invention fulfills these needs, overcomes the deficiencies in prior therapeutic regimen, and provides other related advantages.

### SUMMARY OF THE INVENTION

A first aspect of the present invention relates to a pharmaceutical composition having a stress protein complex, where the stress protein complex includes a stress protein or polypeptide and an immunogenic fungal polypeptide.

A second aspect of the present invention relates to a pharmaceutical composition having a first polynucleotide encoding a stress protein or polypeptide and a second polynucleotide encoding an immunogenic fungal polypeptide.

A third aspect of the present invention relates to a pharmaceutical composition having an antigen presenting cell modified to present a stress protein or polypeptide and an immunogenic fungal polypeptide.

A fourth aspect of the present invention is a method of treating or preventing a fungal disease in a subject. This method involves administering to a subject a pharmaceutical composition according to the first, second, or third aspects of the present invention in an amount effective to induce an immune response against the immunogenic fungal polypeptide in the subject, whereby the immune response treats or prevents the fungal disease in the subject.

A fifth aspect of the present invention relates to a method of treating fungal disease in a subject. This method involves activating antigen presenting cells in vitro with a stress protein or polypeptide, contacting the activated antigen presenting cells with a fungal antigenic peptide, and introducing the contacted and activated antigen presenting cells into a subject having a fungal disease, thereby treating the fungal disease.

A sixth aspect of the present invention relates to a transgenic antigen presenting cell where the cell includes a first polynucleotide encoding a stress protein or polypeptide and a second polynucleotide encoding an immunogenic fungal polypeptide.

The use of a natural, autologous stress protein or polypeptide adjuvant has significant clinical importance, because effective adjuvants and effective therapeutics for fungal diseases are currently lacking. The present invention provides both active vaccines (containing either (i) a human stress protein or polypeptide complexed to a relevant immunogenic fungal polypeptide or (ii) a nucleic acid vaccine that encodes these polypeptides) and passive vaccines (containing activated antigen presenting cells). These vaccines will afford a highly potent, yet safe, antifungal vaccine suitable for prophylaxis and therapy in humans as well as in other animals.

These studies provide a rationale to evaluate strategies that stimulate or inhibit specific classes of TLRs as a means of stimulating immune effector functions to classes of pathogens. Without being bound by belief, it is believed that a candidate vaccine containing HSP-110/Aspergillus antigen may lead to enhanced stimulation of the TLR 4 pathway which, in turn, would be expected to stimulate maturation of dendritic cells, increase antigen presentation, and drive type I cytokine responses. These features should enhance fungal clearance and protect against experimental aspergillosis.

### BRIEF DESCRIPTION OF THE DRAWINGS

FIG. 1 is a graph of flow cytometry analysis showing upregulation of the expression of MHC class II, B7.2, and CD40 molecules in mouse bone marrow derived cells (BMDCs) treated with HSP110 (150 μg/ml), LPS (1 μg/ml) (positive control), or luciferase (80-150 μg/ml). One representative set of data is presented from several experiments. Mouse serum (1 μg/200 μl) was used as isotype control and revealed <15 Mean Fluorescence Intensity (MFI).

FIG. 2 is a Western blot confirming generation of HSP110/Asp f2 stress protein complex. The HSP110/Asp f2 complex was generated under heat shock conditions. Lane A: unbound (1 μg) Asp f2 (37 kDa) was loaded as a control (arrow). Lane B: when incubated with rabbit antiserum (negative control), no bands were present in a Western blot probed with anti-Asp f2 antibody. Lane C: when incubated with anti-HSP110 antiserum, Western blot probed with anti-Asp f2 confirmed the presence of Asp f2.

FIGS. 3A-C show CD86 expression on dendritic cells (DCs) following stimulation with Asp f2, HSP110, HSP110/Asp f2 complex, and LPS (positive control). FIG. 3A shows results with wildtype DCs. FIG. 3B shows results with TLR 4-/-DCs. A comparison of mean fluorescent intensity (MFI) is summarized in FIG. 3C.

FIGS. 4A-B are graphs showing IgG1 and IgG2a in vivo expression, respectively, in mice immunized on day 0 and day 14 with the HSP110/Asp f2 complex or Asp f2 antigen alone. FIG. 4A shows serum IgGI levels at day 25. FIG. 4B shows serum IgG2a levels at day 25. Serum IgGI levels specific for Asp f2 were similar in mice immunized with the HSP110/Asp f2 complex and Asp f2 alone, shown in FIG. 4A. In contrast, serum Asp f2-specific IgG2a levels were approximately 10-fold higher in HSP110/Asp f2 compared to Asp f2 alone recipients (p=0.005), as shown in FIG. 4B.

FIGS. 5A-B are two representative ELISPOT wells showing IFN-γ production from PBMCs from a CGD patient with invasive aspergillosis. FIG. 5A shows adherent cells stimulated overnight with fungal antigen. FIG. 5B shows results when non-adherent cells (containing the lymphocyte responder population) were added.

FIG. 6 is a graph showing morbidity-free survival of CGD mice after intratracheal Aspergillus fumigatus challenge (1.25×10⁴ CFU/mouse) in relation to antifungal therapy. “Amb-d” is amphotericin B treatment; “FK463” is echinocandin FK463 (micafungin) treatment; “Amb-d+FK463” shows cells treated with a combination of the antifungals.

FIGS. 7A-D show the histopathology of Aspergillus infection in CGD mice. FIG. 7A shows foci of inflammation present in the lungs, day 4 following intranasal challenge, hematoxylin and eosin (H&E) staining, 5033 . FIG. 7B shows same sample as FIG. 7A but at higher power magnification (630×), showing neutrophils surrounding a hyphal element (arrow). In FIG. 7C, GMS stain shows invasive hyphae (200×). In FIG. 7D, lung tissue nine weeks after sub-lethal challenge shows that well-defined granulomata persist and hyphae are observable with H&E staining (200×).

### DETAILED DESCRIPTION OF THE INVENTION

The present invention relates to pharmaceutical compositions that can be used as active or passive vaccines for the treatment or prevention of fungal disease.

According to one embodiment, the pharmaceutical composition contains a stress protein complex that includes a stress protein or polypeptide and an immunogenic fungal polypeptide.

The present invention is based on the efficacy of stress proteins or polypeptides to facilitate an effective immune response, providing a basis for their use in presenting a variety of antigens for prophylaxis and therapy of allergic and/or infectious fungal diseases (see PCT Publ. No. WO 01/23421; U.S. Patent Applications Publ. Nos. 20050202035 and 20020039583, all to Subjeck et al., which are hereby incorporated by reference in their entirety).

Within the context of the present invention, stress polypeptides contain at least a peptide binding portion of the full length stress protein and/or a variant thereof. Polypeptides as described herein may be of any length. Additional sequences derived from the native stress protein and/or heterologous sequences may be present, and such sequences may, but need not, possess further peptide binding, immunogenic or antigenic properties.

Suitable stress proteins or polypeptides can be heat shock proteins (“HSP”) or polypeptides, glucose regulated proteins (“GRP”) or polypeptides, or any other stress protein that can facilitate an effective immune response.

Many heat shock proteins are found in the cytoplasm and, to a lesser extent, in the nucleus, and are well known to those in the art. The major families of heat shock proteins include HSP25 (or HSP27 or HSP28), HSP70, HSP90, and HSP110, including all isoforms, analogues, and homologues thereof. Exemplary members of the HSP25/27/28 family include, without limitation, those identified at Genbank Accession Nos. P14602 (mouse), NP_{—}114176 (rat), NP_{—}001531 (human), AAH12768 (human), AAH00510 (human), and AAH12292 (human), each of which and its corresponding nucleic acid accession is hereby incorporated by reference in its entirety. Exemplary members of the HSP70 family include, without limitation, those identified at Genbank Accession Nos. AAC84169 (mouse), NP_{—}034608 (mouse), AAA17441 (rat), Q07439 (rat), NP_{—}002145 (human), AAI10862 (human), AAA02807 (human), and AAA52697 (human), each of which and its corresponding nucleic acid accession is hereby incorporated by reference in its entirety (see also PFAM00012, which is hereby incorporated by reference in its entirety). Exemplary members of the HSP90 family include, without limitation, those identified at Genbank Accession Nos. NP_{—}034610 (mouse), NP_{—}032328 (mouse), NP_{—}786937 (rat), NP_{—}001004082 (rat), P08238 (human), AAI21063 (human), each of which and its corresponding nucleic acid accession is hereby incorporated by reference in its entirety (see also PFAM00183, which is hereby incorporated by reference in its entirety). Exemplary members of the HSP110 family include, without limitation, those identified at Genbank Accession Nos. AAH18378 (mouse), NP_{—}038587 (mouse), Q66H48 (rat), AAH81945 (rat), Q92598 (human), BAA34780 (human), BAA34779 (human), and NP-006635 (human), each of which and its corresponding nucleic acid accession is hereby incorporated by reference in its entirety.

In one aspect of the present invention, the stress protein complex includes HSP110, also known as KIAA0201, NY-CO-25, HSP105 alpha and HSP105 beta. Mouse HSP110 is also known as HSP105 alpha, HSP105 beta, 42° C.-specific heat shock protein, and HSP-E7I. HSP110 is an abundant and strongly inducible mammalian heat shock protein. HSP110 has recently been well-characterized (Morozov et al., “HPV16 E7 Oncoprotein Induces Expression of a 110 kDa Heat Shock Protein,” FEBS Lett 371(3):214-218 (1995); SWISS/Pro Accession Nos. Q61699, Q62578, and Q62579; WO 01/23421 to Subjeck et al.; and U.S. Patent Applications Publ. Nos. 20050202035 and 20020039583 to Subjeck et al., which are hereby incorporated by reference in their entirety).

Functional domains and variants of HSP110 that are capable of mediating the chaperoning and peptide binding activities of HSP110 are identified by Oh et al. (“The Chaperoning Activity of Hsp110: Identification of Functional Domains by Use of Targeted Mutations,” J Biol Chem 274(22):15712-18 (1999), which is hereby incorporated by reference in its entirety). Candidate fragments and variants of the stress polypeptides disclosed herein can be identified as having chaperoning activity by assessing their ability to solubilize heat-denatured luciferase and to refold luciferase in the presence of rabbit reticulocyte lysate (Oh et al., “The Chaperoning Activity of Hsp110: Identification of Functional Domains by Use of Targeted Mutations,” J Biol Chem 274(22):15712-18 (1999), which is hereby incorporated by reference in its entirety).

Glucose regulated proteins (GRPs) reside in the endoplasmic reticulum, and are well known to those of skill in the art. The major families of glucose regulated proteins includes GRP78, GRP94, and GRP170, including all isoforms, analogues, and homologues thereof. This category of stress proteins lack heat shock elements in their promoters and are not inducible by heat, but instead by other stress conditions such as anoxia. Exemplary members of the GRP78 family include, without limitation, those identified at Genbank Accession Nos. NP_{—}071705 (mouse), P20029 (mouse), AAA41277 (rat), AAA51448 (rat), NP_{—}005338 (human), P11021 (human), and AAF13605 (human), each of which and its corresponding nucleic acid accession is hereby incorporated by reference in its entirety. Exemplary members of the GRP94 family include, without limitation, those identified at Genbank Accession Nos. AAH10445 (mouse), AAH11439 (mouse), NP_{—}003290 (human), and AAH66656 (human), each of which and its corresponding nucleic acid accession is hereby incorporated by reference in its entirety. Exemplary members of the GRP170 family include, without limitation, those identified at Genbank Accession Nos. AAF65544 (mouse), AAB35051 (mouse), AAH50107 (mouse), NP_{—}067370 (mouse), Q63617 (rat), AAH65310 (rat), AAB05672 (rat), AAC50947 (human), ABC75106 (human), and ABD14370 (human), each of which and its corresponding nucleic acid accession is hereby incorporated by reference in its entirety.

GRP170 is a strong structural homolog to HSP110 that resides in the endoplasmic reticulum (Lin et al., “The 170-kDa Glucose-Regulated Stress Protein is an Endoplasmic Reticulum Protein that Binds Immunoglobulin,” Mol Biol Cell 4:1109-19 (1993); Chen et al., “The 170 kDa Glucose Regulated Stress Protein is a Large HSP70-HSP110-Like Protein of the Endoplasmic Reticulum,” FEBS Lett 380:68-72 (1996); Dierks et al., “A Microsomal ATP-Binding Protein Involved in Efficient Protein Transport Into the Mammalian Endoplasmic Reticulum,” EMBO J. 15:6931-42 (1996), which are hereby incorporated by reference in their entirety). Functional domains of GRP170 parallel those of HSP110. GRP170 is also known as ORP150 (oxygen-regulated protein identified in both human and rat) and as CBP-140 (calcium binding protein identified in mouse). GRP170 has been shown to stabilize denatured protein more efficiently than HSP70.

Stress protein complexes of the invention can be obtained through a variety of methods. In one example, a recombinant HSP110 or GRP170 is mixed with cellular material (e.g., lysate), to permit binding of the stress polypeptide with one or more immunogenic polypeptides within the cellular material. Such binding can be enhanced or altered by stress conditions, such as heating of the mixture. In another example, target cells are transfected with HSP110 or GRP170 that has been tagged (e.g., HIS tag) for later purification. In yet another example, heat or other stress conditions are used to induce HSP110 or GRP170 in target cells prior to purification of the stress polypeptide. This stressing can be performed in situ, in vitro, or in cell cultures.

In some embodiments, the present invention provides a stress protein complex having enhanced immunogenicity that includes a stress polypeptide and an immunogenic polypeptide, wherein the complex has been heated. Such heating, particularly wherein the stress polypeptide is a heat-inducible stress protein, can increase the efficacy of the stress protein complex as a vaccine. Examples of heat-inducible stress proteins include the above-identified HSPs. In one embodiment, heating involves exposing tissue including the stress protein complex to a temperature of at least approximately 38° C., and then gradually increasing the temperature, e.g. by 1° C./10 min. until the desired level of heating is obtained. Preferably, the temperature of the tissue is brought to approximately 39.5° C.±0.5° C. At the time of heating, the tissue can be in vivo, in vitro, or positioned within a host environment.

The immunogenic polypeptides of the present invention include fungal antigen suitable for complexing with a stress protein or polypeptide, preferably HSP110 or GRP170. The immunogenic polypeptides (i.e., antigens) can be derived from any fungus that is a causative agent of a disease or disease condition in animals, preferably mammals, including but not limited to humans.

In one aspect of present invention the immunogenic peptide of the stress protein complex is a fungal antigen from a member of the genus Aspergillus including, without limitation: A. fumigatus, A. flavus, A. niger, A. clavatus, A. glaucus group, A. nidulans, A. oryzae, A. terreus, A. ustus, and A. versicolor. Also suitable are fungal antigens of Candida spp., Cryptococcus spp., dimorphic fungi, Pneumocystis jirovecii, and non-Aspergillus filamentous fungi. In a preferred embodiment the immunogenic peptide is an A. fumigatus antigen. The genome of A. fumigatus has been sequenced and is publicly available (GenBank Accession Nos. NC_{—}007194-007201, which is hereby incorporated by reference in their entirety). An exemplary list of aspergillus antigens suitable for the present invention is shown in Table 1 below.

TABLE 1
List of Exemplary Aspergillus Antigens

| Organism | Protein | Accession No. or Citation |
|---|---|---|
| A. fumigatus | Asp f1 | XP-748109 |
| A. fumigatus | Asp f2 | AAC69357 |
| | | EAL89830 |
| A. fumigatus | Asp f3 | XP-747849 |
| A. fumigatus | Asp f4 | XP-749515 |
| | | EAL87477 |
| A. fumigatus | Asp f6 | Schwienbacher et al., |
| | | Allergy 60: 1430-1435 |
| | | (2005) |
| A. fumigatus | Asp f9 | CAA 11266 |
| A. fumigatus | Asp f7 | XP-752159 |
| A. fumigatus | Asp f16 | Ramadan et al., Clin |
| | | Exp Immunol 140(1): 81-91 |
| | | (2005) |
| A. fumigatus | Asp f13 | XP-755595 |
| | | EAL93557 |
| A. fumigatus | Putative allergen | EAL86578 |
| | | EAL86354 |
| A. fumigatus | Putative allergen | AAC61261 |

Each of the above-identified accessions or references is hereby incorporated by reference in its entirety.

All of the antigens recited herein, including those currently known in the art, and those characterized in the future as either infectious or allergic fungal antigenic peptides, are encompassed in this and all aspects of the present invention.

A stress protein complex of the invention can also include a variant of a native stress protein and a variant of an immunogenic peptide. A polypeptide “variant,” as used herein, is a polypeptide that differs from a native stress protein in one or more substitutions, deletions, additions and/or insertions, such that the immunogenicity of the polypeptide is not substantially diminished. In other words, the ability of a variant to react with antigen-specific antisera may be enhanced or unchanged, relative to the native protein, or may be diminished by less than 50%, and preferably less than 20%, relative to the native protein. Such variants may generally be identified by modifying one of the above polypeptide sequences and evaluating the reactivity of the modified polypeptide with antigen-specific antibodies or antisera as described herein. It is also preferable that the stress polypeptide variant possesses comparable peptide-binding activity (described above). Preferred variants include those in which one or more portions, such as an N-terminal leader sequence or transmembrane domain, have been removed. Other preferred variants include variants in which a small portion (e.g., 1-30 amino acids, preferably 5-15 amino acids) has been removed from the N- and/or C-terminal of the mature protein.

Polypeptide variants preferably exhibit at least about 70%, more preferably at least about 90% and most preferably at least about 95% identity to the identified polypeptides. Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually about 30 to about 75, or about 40 to about 50, in which a sequence may be compared to a reference sequence of the same number of contiguous positions after the two sequences are optimally aligned.

Optimal alignment of sequences for comparison may be conducted using the MegAlign program in the LaserGene suite of bioinformatics software (DNASTAR, Inc., Madison, Wis.) using default parameters. This program embodies several alignment schemes described in the following references: Dayhoff, M. O., “A Model of Evolutionary Change in Proteins—Matrices for detecting distant relationships,” In Dayhoff, M. O. (ed.) Atlas ofProtein Sequence and Structure, National Biomedical Research Foundation, Washington D.C. Vol. 5, Suppl. 3, pp. 345-358 (1978); Hein J., “Unified Approach to Alignment and Phylogenes,” Methods in Enzymology 183:626-645, Academic Press, Inc., San Diego, Calif. (1990); Higgins et al., “Fast and Sensitive Multiple Sequence Alignments on a Microcomputer,” CABIOS 5:151-153 (1989); Myers et al., “Optimal Alignments in Linear Space,” CABIOS 4:11-17 (1988); Robinson, E. D., Comb. Theor. 11:105(1971); Santou et al., “The Neighbor-Joining Method: A New Method for Reconstructing Phylogenetic Trees,” Mol. Biol. Evol. 4:406-425(1987); Sneath et al., Numerical Taxonomy the Principles and Practice of Numerical Taxonomy, Freeman Press, San Francisco, Calif. (1973); Wilbur et al., “Rapid Similarity Searches of Nucleic Acid and Protein Data Banks,” Proc. Natl. Acad. Sci. USA 80:726-730 (1983), each of which is hereby incorporated by reference in its entirety.

Preferably, the “percentage of sequence identity” is determined by comparing two optimally aligned sequences over a window of comparison of at least 20 positions, wherein the portion of the polynucleotide or polypeptide sequence in the comparison window may comprise additions or deletions (i.e. gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e., the window size) and multiplying the results by 100 to yield the percentage of sequence identity.

Preferably, a variant contains conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. In a preferred embodiment, variant polypeptides differ from a native sequence by substitution, deletion or addition of five amino acids or fewer. Variants may also (or alternatively) be modified by, for example, the deletion or addition of amino acids that have minimal influence on the immunogenicity, secondary structure and hydropathic nature of the polypeptide.

Polypeptides may also include a signal (or leader) sequence at the N-terminal end of the protein that co-translationally or post-translationally directs transfer of the protein. The polypeptide may also be conjugated to a linker or other sequence for ease of synthesis, purification or identification of the polypeptide (e.g., poly-FEs), or to enhance binding of the polypeptide to a solid support. For example, a polypeptide may be conjugated to an immunoglobulin Fc region.

Polypeptides may be prepared using any of a variety of well known techniques (e.g., PCT Publ. No. WO 01/23421; U.S. Patent Applications Publ. Nos. 20050202035 and 20020039583, all to Subjeck et al., which are hereby incorporated by reference in their entirety). In one embodiment, the stress polypeptide(s) and immunogenic polypeptide(s) are co-purified from cells infected with a pathogen as a result of the purification technique. In some embodiments, the tumor cells or infected cells are stressed prior to purification to enhance binding of the immunogenic polypeptide to the stress polypeptide. For example, the cells can be stressed in vitro by several hours of low-level heating (39.5-40° C.) or about 1 to about 2 hours of high-level heating (approximately 43° C.). In addition, the cells can be stressed in vitro by exposure to anoxic and/or ischemic or proteotoxic conditions.

In one embodiment of the present invention, the polypeptide is a fusion protein that contains multiple polypeptides as described herein, or that contains at least one polypeptide as described herein and an unrelated sequence. In one embodiment, the fusion protein includes a stress polypeptide of HSP110 and/or GRP170 and an immunogenic polypeptide. The immunogenic polypeptide can include all or a portion of a protein associated with an allergic or infectious fungal disease.

Additional fusion partners can be added. A fusion partner may, for example, serve as an immunological fusion partner by assisting in the provision of T helper epitopes, preferably T helper epitopes recognized by humans. As another example, a fusion partner may serve as an expression enhancer, assisting in expressing the protein at higher yields than the native recombinant protein. Certain preferred fusion partners are both immunological and expression enhancing fusion partners. Other fusion partners may be selected so as to increase the solubility of the protein or to enable the protein to be targeted to desired intracellular compartments. Still further fusion partners include affinity tags, which facilitate purification of the protein.

Fusion proteins may generally be prepared using standard techniques, including chemical conjugation. A fusion protein may be expressed as a recombinant protein, allowing the production of increased levels, relative to a non-fused protein, in an expression system. Thus, the present invention also relates to a pharmaceutical composition having a first polypeptide encoding an HSP110 or GRP170 polypeptide and a second polynucleotide encoding an immunogenic polypeptide. In a preferred embodiment, the immunogenic polypeptide is a fungal antigen. This involves recombinant molecular biology techniques well known in the art. Briefly, DNA sequences encoding the polypeptide components may be assembled separately, and ligated into an appropriate expression vector. “Vector” is used herein to mean any genetic element, such as a plasmid, phage, transposon, cosmid, chromosome, virus, virion, etc., which is capable of replication when associated with the proper control elements and which is capable of transferring gene sequences between cells. Thus, the term includes cloning and expression vectors, as well as viral vectors. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion protein that retains the biological activity of both component polypeptides.

A peptide linker sequence may be employed to separate the first and the second polypeptide components by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn, and Ser residues. Other near neutral amino acids, such as Thr and Ala, may also be used in the linker sequence. Amino acid sequences which may be usefully employed as linkers include those disclosed in Maratea et al., “Deletion and Fusion Analysis of the Phase phi X174 Lysis Gene E,” Gene 40:39-46 (1985); Murphy et al., “Genetic Construction, Expression, and Melanoma-Selective Cytotoxicity of a Diphtheria Toxin-Related α-Melonocyte-Stimulating Hormone Fusion Protein,” Proc. Natl. Acad. Sci. USA 83:8258-8262 (1986); U.S. Pat. No. 4,935,233 and U.S. Pat. No. 4,751,180, which are all hereby incorporated by reference in their entirety. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements to provide expression of the desired DNA. The regulatory elements responsible for transcription of DNA are located 5′ to the DNA sequence encoding the first polypeptides. Similarly, stop codons required to end translation and transcription termination signals are present 3′ to the DNA sequence encoding the second polypeptide.

In general, polypeptides (including fusion proteins) and polynucleotides as described herein are isolated. An “isolated” polypeptide or polynucleotide is one that is removed from its cellular environment. For example, a naturally occurring protein is isolated if it is separated from some or all of the coexisting materials in the natural system. A polynucleotide is considered to be isolated if, for example, it is cloned into a vector that is not a part of the natural environment.

Preferably, such polypeptides are at least about 90% pure, more preferably at least about 95% pure and most preferably at least about 99% pure.

According to a second embodiment, the pharmaceutical composition includes a first polynucleotide encoding a stress protein or polypeptide, and a second polynucleotide encoding an immunogenic protein or polypeptide that is a fungal antigen. In this aspect of the present invention, the first polynucleotide encodes any of the above-described stress proteins, but preferably HSP110 or GRP170 or a portion or other variant thereof, and the second polynucleotide encodes any one or more of the above-described immunogenic polypeptides, or a portion or other variant thereof, but preferably a fungal antigen.

In some embodiments, the first and second polynucleotides are operatively coupled to form a single polynucleotide that encodes a stress protein complex. The single polynucleotide can express the first and second proteins in a variety of ways, for example, as a single fusion protein or as two separate proteins capable of forming a complex. Preferred polynucleotides comprise at least 15 consecutive nucleotides, preferably at least 30 consecutive nucleotides and more preferably at least 45 consecutive nucleotides, which encode a portion of a stress protein or immunogenic polypeptide. More preferably, the first polynucleotide encodes a peptide binding portion of a stress protein and the second polynucleotide encodes an immunogenic portion of an immunogenic polypeptide. Polynucleotides complementary to any such sequences are also encompassed by the present invention. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

Polynucleotides may comprise a native sequence (i.e., an endogenous sequence that encodes a stress protein, immunogenic polypeptide or a portion thereof) or may comprise a variant of such a sequence. Polynucleotide variants contain one or more substitutions, additions, deletions and/or insertions such that the immunogenicity of the encoded polypeptide is not diminished, relative to a native stress protein. The effect on the immunogenicity of the encoded polypeptide may generally be assessed as described herein. Variants preferably exhibit at least about 70% identity, more preferably at least about 80% identity and most preferably at least about 90% identity to a polynucleotide sequence that encodes a native stress protein or a portion thereof.

Variants may also, or alternatively, be substantially homologous to a native gene, or a portion or complement thereof. Such polynucleotide variants are capable of hybridizing under moderately stringent conditions to a naturally occurring DNA sequence encoding a native stress protein (or its complementary sequence). Suitable moderately stringent conditions include prewashing in a solution of 5×SSC, 0.5% SDS, 1.0 mM EDTA (pH 8.0); hybridizing at 50 C.-65° C., 5×SSC, overnight; followed by washing twice at 65° C. for 20 minutes with each of 2×SSC, 0.5×SSC, and 0.2×SSC containing 0.1% SDS. Lowering the sodium content and increasing the temperature can be used to enhance the stringency of hybridization conditions.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

Polynucleotides may be prepared using any of a variety of techniques known in the art. DNA encoding a stress protein may be obtained from a cDNA library prepared from tissue expressing a stress protein mRNA. Accordingly, a human stress protein-encoding polynucleotide, such as human HSP110 or GRP170 DNA, can be conveniently obtained from a cDNA library prepared from human tissue. The stress protein-encoding gene may also be obtained from a genomic library or by oligonucleotide synthesis. Libraries can be screened with probes (such as antibodies to the stress protein or oligonucleotides of at least about 20-80 bases) designed to identify the gene of interest or the protein encoded by it. Illustrative libraries include human liver cDNA library (human liver 5′ stretch plus cDNA, Clontech Laboratories, Inc.) and mouse kidney cDNA library (mouse kidney 5′-stretch cDNA, Clontech Laboratories, Inc.). Screening the cDNA or genomic library with the selected probe may be conducted using standard procedures, such as those described in Sambrook et al., Molecular Cloning: A Laboratory Manual (New York: Cold Spring Harbor Laboratory Press (1989), which is hereby incorporated by reference in its entirety). An alternative means to isolate the gene encoding HSP110 or GRP170, or any other stress protein, is to use PCR methodology (Sambrook et al., Molecular Cloning: A Laboratory Manual (New York: Cold Spring Harbor Laboratory Press (1989); Dieffenbach et al., PCR Primer: A Laboratory Manual Cold Spring Harbor Laboratory Press (1995), which is hereby incorporated by reference in its entirety).

The oligonucleotide sequences selected as probes should be sufficiently long and sufficiently unambiguous that false positives are minimized. The oligonucleotide is preferably labeled such that it can be detected upon hybridization to DNA in the library being screened. Methods of labeling are well known in the art, and include the use of radiolabels, such as ³²P-labeled ATP, biotinylation, enzyme labels, or fluorescent labels. Hybridization conditions, including moderate stringency and high stringency, are as provided in Sambrook et al., Molecular Cloning: A Laboratory Manual (New York: Cold Spring Harbor Laboratory Press (1989), which is hereby incorporated by reference in its entirety), or as known in the art.

Sequences identified in such library screening methods can be compared and aligned to other known sequences deposited and available in public databases such as GenBank or other private sequence databases. Sequence identity (at either the amino acid or nucleotide level) within defined regions of the molecule or across the full-length sequence can be determined through sequence alignment using computer software programs, which employ various algorithms to measure homology as described above.

Nucleic acid molecules having protein coding sequence may be obtained by screening selected cDNA or genomic libraries, and, if necessary, using conventional primer extension procedures as described in Sambrook et al., Molecular Cloning: A Laboratory Manual (New York: Cold Spring Harbor Laboratory Press (1989) (which is hereby incorporated by reference in its entirety), to detect precursors and processing intermediates of mRNA that may not have been reverse-transcribed into cDNA.

Polynucleotide variants may generally be prepared by any method known in the art, including chemical synthesis by, for example, solid phase phosphoramidite chemical synthesis. Modifications in a polynucleotide sequence may also be introduced using standard mutagenesis techniques, such as oligonucleotide-directed site-specific mutagenesis (Adelman et al., DNA 2:183 (1983), which is hereby incorporated by reference in its entirety). Alternatively, RNA molecules may be generated by in vitro or in vivo transcription of DNA sequences encoding a stress protein, or portion thereof, provided that the DNA is incorporated into a vector with a suitable RNA polymerase promoter (such as T7 or SP6). Certain portions may be used to prepare an encoded polypeptide, as described herein. In addition, or alternatively, a portion may be administered to a patient such that the encoded polypeptide is generated in vivo (e.g., by transfecting antigen-presenting cells, such as dendritic cells, with a cDNA construct encoding a stress polypeptide, and administering the transfected cells to the patient).

Any polynucleotide may be further modified to increase stability in vivo. Possible modifications include, but are not limited to, the addition of flanking sequences at the 5′ and/or 3′ ends; the use of phosphorothioate or 2′ O-methyl rather than phosphodiesterase linkages in the backbone; and/or the inclusion of nontraditional bases such as inosine, queosine and wybutosine, as well as acetyl- methyl-, thio- and other modified forms of adenine, cytidine, guanine, thymine and uridine.

According to a third embodiment, the pharmaceutical composition includes an antigen presenting cell modified to present a stress protein or polypeptide as described above, preferably HSP110 or GRP170, and an immunogenic polypeptide as described above, preferably a fungal antigen.

As used herein, “antigen-presenting cell” (APC) includes any cell capable of handling and presenting antigen to a lymphocyte. Examples of APCs include, without limitation, macrophages, Langerhans-dendritic cells, follicular dendritic cells, B cells, monocytes, fibroblasts and fibrocytes. Dendritic cells are a preferred type of antigen presenting cell for use in the present invention. Dendritic cells are found in many non-lymphoid tissues but can migrate via the afferent lymph or the blood stream to the T-dependent areas of lymphoid organs. In non-lymphoid organs, dendritic cells include Langerhans cells and interstitial dendritic cells. In the lymph and blood, they include afferent lymph veiled cells and blood dendritic cells, respectively. In lymphoid organs, they include lymphoid dendritic cells and interdigitating cells.

In this aspect of the present invention, suitable APCs include cells that have been modified to present an epitope. This refers to APCs that have been manipulated to present an epitope by natural or recombinant methods. For example, the APCs can be modified by exposure to the isolated antigen, alone or as part of a mixture, peptide loading, or by genetically modifying the APC to express a polypeptide that includes one or more epitopes.

Some embodiments of the present invention use dendritic cells or progenitors thereof as antigen-presenting cells. Dendritic cells are highly potent APCs (Banchereau et al., “Dendritic Cells and the Control of Immunity,” Nature 392:245-251 (1998), which is hereby incorporated by reference in its entirety) and have been shown to be effective as a physiological adjuvant for eliciting prophylactic or therapeutic antitumor immunity (Timmerman et al., “Dendritic Cell Vaccines for Cancer Immunotherapy,” Annu Rev Med 50:507-529 (1999), which is hereby incorporated by reference in its entirety). In general, dendritic cells may be identified based on their typical shape (stellate in situ, with marked cytoplasmic processes (dendrites) visible in vitro) and based on the lack of differentiation markers of B cells (CD19 and CD20), T cells (CD3), monocytes (CD 14), and natural killer cells (CD56), as determined using standard assays. Dendritic cells may be engineered to express specific cell surface receptors or ligands that are not commonly found on dendritic cells in vivo or ex vivo, and such modified dendritic cells are encompassed by the present invention. As an alternative to dendritic cells, secreted vesicles antigen-loaded dendritic cells (called exosomes) may be used within a vaccine (see Zitvogel et al., “Eradication of Established Murine Tumors Using a Novel Cell-Free Vaccine: Dendritic Cell-Derived Exosomes,” Nature Med 4:594-600 (1998), which is hereby incorporated by reference in its entirety).

Dendritic cells and progenitors may be obtained from peripheral blood, bone marrow, tumor-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNF-α to cultures of monocytes harvested from peripheral blood. Alternatively, CD34 positive cells harvested from peripheral blood, umbilical cord blood, or bone marrow may be differentiated into dendritic cells by adding to the culture medium combinations of GM-CSF, IL-3, TNF-α, CD40 ligand, LPS, flt3 ligand and/or other compound(s) that induce maturation and proliferation of dendritic cells. Dendritic cells are conveniently categorized as “immature” and “mature” cells, which allows a simple way to discriminate between two well characterized phenotypes. However, this nomenclature should not be construed to exclude all possible intermediate stages of differentiation. Immature dendritic cells are characterized as APC with a high capacity for antigen uptake and processing, which correlates with the high expression of Fcy receptor, mannose receptor, and DEC-205 marker. The mature phenotype is typically characterized by a lower expression of these markers, but a high expression of cell surface molecules responsible for T cell activation such as class I and class II MHC, adhesion molecules (e.g., CD54 and CD11), and costimulatory molecules (e.g., CD40, CD80, and CD86).

APCs may generally be transfected with a polynucleotide encoding a stress protein (or portion or other variant thereof) such that the stress polypeptide, or an immunogenic portion thereof, is expressed on the cell surface. Such transfection may take place ex vivo, and a composition or vaccine comprising such transfected cells may then be used for therapeutic purposes, as described herein.

Alternatively, a gene delivery vehicle that targets a dendritic or other antigen presenting cell may be administered to a patient, resulting in transfection that occurs in vivo. In vivo and ex vivo transfection of dendritic cells, for example, may generally be performed using any methods known in the art, such as those described in WO 97/24447, or the gene gun approach described by Mahvi et al., “DNA Cancer Vaccines: A Gene Gun Approach,” Immunology and Cell Biology 75:456-460 (1997), which is hereby incorporated by reference in its entirety). Antigen loading of dendritic cells may be achieved by incubating dendritic cells or progenitor cells with the stress polypeptide, DNA (naked or within a plasmid vector) or RNA; or with antigen-expressing recombinant bacterium or viruses (e.g., vaccinia, fowlpox, adenovirus or lentivirus vectors). Prior to loading, the polypeptide may be covalently conjugated to an immunological partner that provides T cell help (e.g., a carrier molecule). Alternatively, a dendritic cell may be exposed to or contacted (“pulsed”) with a non-conjugated immunological partner, separately or in the presence of the polypeptide.

Administration can be accomplished by a single direct injection at a single time point or multiple time points to a single site or multiple sites. Administration can also be nearly simultaneous to multiple sites. Effector cells may generally be obtained in sufficient quantities for adoptive immunotherapy by growth in vitro, as described herein. Culture conditions for expanding single antigen-specific effector cells to several billion in number with retention of antigen recognition in vitro are well known in the art. Such in vitro culture conditions typically use intermittent stimulation with antigen, often in the presence of cytokines (e.g., IL-2) (Cheever et al., “Therapy with Cultured T Cells: Principles Revisited,” Immunological Reviews 157:177 (1997), which is hereby incorporated by reference in its entirety) and non-dividing feeder cells. Immunoreactive polypeptides as provided herein may be used to rapidly expand antigen-specific T cell cultures to generate a sufficient number of cells for immunotherapy.

Alternatively, a vector expressing any stress protein or polypeptide described above can be introduced into antigen presenting cells taken from a patient and clonally propagated ex vivo for transplant back into the same patient. Transfected cells may be reintroduced into the patient using any means known in the art, preferably in sterile form by intravenous, intracavitary, or intraperitoneal administration. In this aspect of the present invention, the immunogenic polypeptide of the pharmaceutical composition is a fungal antigen as described above.

The pharmaceutical compositions of the present invention may include a suitable pharmaceutically acceptable carrier for administration. As used herein, “pharmaceutically acceptable carrier” includes any material which, when combined with an active ingredient, allows the ingredient to retain biological activity and is non-reactive with the subject's immune system. Examples include, but are not limited to, any of the standard pharmaceutical carriers, such as a phosphate buffered saline solution, water, emulsions such as oil/water emulsion, and various types of wetting agents. Preferred diluents for aerosol or parenteral administration are phosphate buffered saline or normal (0.9%) saline.

Compositions containing such carriers are formulated by well known conventional methods (see, e.g., Remington's Pharmaceutical Sciences, Chapter 43, 14th Ed., Mack Publishing Co, Easton, Pa., USA, which is hereby incorporated by reference in its entirety).

The pharmaceutical compositions of the present invention may also include an adjuvant for enhancing the immunogenic efficacy of the composition when administered to a suitable subject. As used herein, “adjuvant” includes those adjuvants commonly used in the art to facilitate an immune response. Examples of suitable adjuvants include, without limitation, helper peptide; aluminum salts such as aluminum hydroxide gel (alum) or aluminum phosphate; Freund's Incomplete Adjuvant and Complete Adjuvant (Difco Laboratories, Detroit, Mich.); Merck Adjuvant 65 (Merck and Company, Inc., Rahway, N.J.); AS-2 (Smith-Kline Beecham); QS-21 (Aquilla Biopharmaceuticals); MPL or 3d-MPL (Corixa Corporation, Hamilton, Mont.); LEIF; salts of calcium, iron or zinc; an insoluble suspension of acylated tyrosine; acylated sugars; cationically or anionically derivatized polysaccharides; polyphosphazenes; biodegradable nucrospheres; monophosphoryl lipid A and quil A; muramyl tripeptide phosphatidyl ethanolamine; or an immunostimulating complex, including cytokines (e.g., GM-CSF or interleukin-2, -7 or -12) and immunostimulatory DNA sequences. In some embodiments, such as with the use of a polynucleotide vaccine, an adjuvant such as a helper peptide or cytokine can be provided via a polynucleotide encoding the adjuvant.

The pharmaceutical compositions can also include, or be administered in combination with, a compound that has anti-fungal activity, e.g., amphotericin B, voriconazole, FK463, etc.

The pharmaceutical compositions of the present invention are suitable for the treatment of fungal diseases. Treatment, as used herein, includes both prophylaxis and therapy. With regard to therapy, it is intended that administration of the pharmaceutical compositions can be used either to rid a patient of a particular fungal pathogen or diminish the population level of the fungal pathogen to normal levels (i.e., overcome the infection). With regard to prophylaxis, the pharmaceutical composition can be administered prior to or simultaneous with a therapy for a distinct condition, which therapy is known to induce opportunistic fungal infections. In this case, the pharmaceutical composition of the present invention can either completely prevent fungal infection or limit the severity thereof. Therefore, another aspect of the present invention relates to a method of treating or preventing a fungal disease in a subject by administering an effective amount of a pharmaceutical composition of the present invention to a subject, thereby treating or preventing a fungal disease in the subject.

In one aspect of the present invention, prevention or treatment of the fungal disease is carried out as active immunotherapy, in which case treatment relies on the in vivo stimulation of the endogenous host immune system to react against infected cells with the administration of immune response-modifying agents, i.e, the immunogenic polypeptides of the present invention. This method involves administering to a subject an effective amount of a pharmaceutical composition of the present invention. Administration of the pharmaceutical composition to a subject induces the requisite immunogenic response against the fungal pathogen, and thereby prevents or treats the fungal disease in the subject. The pharmaceutical composition is meant to encompass all compositions of stress proteins or polypeptides and immunogenic fungal antigens described above, as well as polynucleotide-based compositions as described above.

In another aspect of the invention, prevention or treatment of the fungal disease is carried out using adoptive immunotherapy, where the treatment involves the delivery of agents with established fungal-antigen reactivity, which can directly or indirectly mediate an anti-fungal effect that does not necessarily depend on an intact immune system. Generally, this method involves activating antigen presenting cells by treating the cells with a heat shock protein in vitro, and pulsing (i.e., contacting) the activated APCs with a peptide of interest. Antigen presenting cells in this aspect of the present invention are as described herein above in all aspects and features. In this embodiment, the APCs are preferably dendritic cell and macrophages. In a preferred embodiment, dendritic cells are modified in vitro to present the polypeptide, and these modified APCs are administered to the subject. T cell receptors and antibody receptors specific for the polypeptides recited herein may be cloned, expressed, and transferred into other vectors or effector cells for adoptive immunotherapy. The polypeptides provided herein may also be used to generate antibodies or anti-idiotypic antibodies (as described above and in U.S. Pat. No. 4,918,164, which is hereby incorporated by reference in its entirety) for passive immunotherapy.

The present invention also relates to a method of treatment of a fungal disease where antigen-presenting cells, such as dendritic, macrophage, monocyte, fibroblast and/or B cells, are pulsed with immunoreactive polypeptides or transfected with one or more polynucleotides using standard techniques well known in the art. For example, antigen-presenting cells can be transfected with a polynucleotide having a promoter appropriate for increasing expression in a recombinant virus or other expression system. Cultured effector cells for use in therapy must be able to grow and distribute widely, and to survive long term in vivo. Cultured effector cells can be induced to grow in vivo and to survive long term in substantial numbers by repeated stimulation with antigen supplemented with IL-2 (Cheever et al., Immunological Reviews 157:177 (1997) which is hereby incorporated by reference in its entirety).

Alternatively, a vector expressing an immunogenic fungal polypeptide as described herein above can be introduced into antigen presenting cells taken from a patient and clonally propagated ex vivo for transplant back into the same patient. Transfected cells may be reintroduced into the patient using any means known in the art, preferably in sterile form by intravenous, intracavitary, intraperitoneal, or intratumoral administration.

Routes and frequency of administration of the therapeutic compositions disclosed herein, as well as dosage, will vary from individual to individual, and may be readily established using standard techniques. In general, the pharmaceutical compositions and vaccines may be administered by injection, e.g., intracutaneous, intratumoral, intramuscular, intravenous or subcutaneous; intranasally (e.g., by aspiration) or orally. They may be administered alone or with suitable pharmaceutical carriers, and can be in solid or liquid form, such as tablets, capsules, powders, solutions, suspensions, or emulsions.

The pharmaceutical compounds of the present invention may also be administered directly to the airways in the form of an aerosol. For use as aerosols, the compounds in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

A suitable (“effective”) dose of a pharmaceutical composition of the present invention, is an amount that, when administered as described above, is capable of promoting an anti-fungal immune response, and is at least 10-50% above the basal (i.e., untreated) level. Such response can be monitored, for example, by measuring the anti-fungal antibodies present in serum withdrawn from the patient undergoing treatment. Such vaccines should also be capable of causing an immune response that leads to an improved clinical outcome (e.g., complete or partial or longer disease-free survival) in vaccinated patients as compared to non-vaccinated patients.

The dose administered to a patient, in the context of the present invention, should be sufficient to induce a beneficial therapeutic response in the patient over time, or to inhibit infection or disease due to infection. Thus, the composition is administered to a subject in an amount sufficient to elicit an effective immune response to the specific antigens and/or to alleviate, reduce, cure or at least partially arrest symptoms and/or complications from the disease or infection. An amount adequate to accomplish this is defined as an “effective” or “therapeutically effective” dose. This aspect of the present invention also encompasses repeated administration of a pharmaceutical composition of the present invention to achieve a suitably effective response in the subject.

Also encompassed in the present invention are methods that include the administration of more than one of the pharmaceutical compositions of the present invention to a subject for the prevention or treatment of a fungal disease. By administering a combination of the pharmaceutical compositions of the present invention to a subject, more than one pathway of immunity may be triggered in the subject, thereby eliciting multiple immune responses in the subject.

Fungal diseases suitable for prevention and treatment according to the present invention include infectious fungal diseases. Examples of such diseases are, without limitation, invasive aspergillosis, which can involve infection of the lungs, sinus cavities, kidneys, central nervous system, and other organs and tissues, leading to pulmonary aspergillosis (the leading form of invasive aspergillosis), cutaneous aspergillosis, hepatosplenic aspergillosis, Aspergillus fungemia, disseminated aspergillosis, onychomycosis, sinusitis, cerebral meningitis, endocarditis, myocarditis, osteomyelitis, otomycosis, endophthalmitis, and nosocomial aspergillosis (due to catheters and other devices). Also included in this aspect are disease conditions due to presence of a secondary fungal infection. For example, Aspergillus spp. may be local colonizers in previously developed lung cavities due to tuberculosis, sarcoidosis, bronchiectasis, pneumoconiosis, ankylosing spondylitis or neoplasms, presenting as a distinct clinical entity, called aspergilloma. Subjects most susceptible are those that are immunocompromised for any reason, for example: AIDS patients, those undergoing cancer treatment, burn patients, or individuals with chronic granulomatous disease.

Fungi are known to cause infections in many species of mammals, including man. Thus, suitable subjects for the methods of prevention and treatment of fungal disease according to the present invention are mammals, including, without limitation, human, bovine, equine, canine, feline, porcine, and ovine animals. The subject is preferably a human, and may or may not be afflicted with a disease.

The methods of the present invention also relate to prevention and treatment of fungal diseases that result from the allergens of fungal organisms, for example, allergic bronchopulmonary aspergillosis (ABPA), particularly in atopic individuals (Banerjee et al., “Purification of a Major Allergen, Asp f 2 Binding to IgE in Allergic Bronchopulmonary Aspergillosis, From Culture Filtrate of Aspergillus Fumigatus,” J Allergy Clin Immunol 99:821-7 (1997); Banerjee et al., “Molecular Cloning and Expression of a Recombinant Aspergillus Fumigatus Protein Asp fII With Significant Immunoglobulin E Reactivity in Allergic Bronchopulmonary Aspergillosis,” J Lab Clin Med 127:253-62 (1996); Banerjee et al., “Immunological Characterization of Asp f2, a Major Allergen From Aspergillus Fumigatus Associated With Allergic Bronchopulmonary Aspergillosis” Infect Immun 66:5175-82(1998); Banerjee et al., “Conformational and Linear B-Cell Epitopes of Asp f2, a Major Allergen of Aspergillus Fumigatus, Bind Differently to Immunoglobulin E Antibody in the Sera of Allergic Bronchopulmonary Aspergillosis Patients,” Infect Immun 67:2284-91 (1999), which are hereby incorporated by reference in their entirety).

Other fungal respiratory diseases suitable for treatment using the pharmaceutical compositions of the present invention include hypersensitivity pneumonitis, allergic asthma, and respiratory aspergilloma.

### EXAMPLES

### Example 1

### HSP110 Matures Dendritic Cells

Heat shock proteins are a ubiquitous group of intracellular molecules that function as molecular chaperones in numerous processes such as protein folding, assembly, transport, and peptide trafficking and antigen processing (Manjili et al., “Immunotherapy of Cancer Using Heat Shock Proteins,” Front Biosci 7:d43-52 (2002); Manjili et al., “Cancer Immunotherapy: Stress Proteins and Hyperthermia,” Int J Hyperthermia 18:506-20 (2002), which are hereby incorporated by reference in their entirety). They are induced by several environmental stressors, such as fever, oxidative stress, alcohol, inflammation, and heavy metals. HSP expression is also induced by conditions associated with injury and necrosis, including infection, trauma, and ischemic reperfusion injury. During such periods of physiologic stress, HSPs bind to exposed hydrophobic sites within polypeptides and mediate conformational changes, prevent misfolding of peptides, and facilitate peptide transport across membranes. Thus, different groups of HSPs have diverse regulatory functions during physiologic stress and injury. Moreover, HSPs are potent inducers of innate and antigen-specific immunity. Their role as “danger signals” that prime multiple host defense pathways are being exploited in vaccine development in cancer.

For many years, HSPs have been considered to be exclusively intracellular proteins with intracellular functions and their appearance outside of the cell to be artifacts, e.g., due to cell lysis. Recently, this view has changed. Cell damage is no longer considered an artifact, but to have essential functions in alarming the host to damaged or diseased tissues. The activation of DCs, necessary for the initiation of primary and secondary immune responses, can be induced by motifs present on pathogens (e.g., endotoxin) as well as endogenous danger signals released by tissues undergoing stress, damage or necrosis. Examples of endogenous danger signals include HSPs, nucleotides, reactive oxygen intermediates, extracellular matrix breakdown products, neuromediators and cytokines like the interferons (Gallucci et al., “Danger Signals: SOS to the Immune System,” Curr Opin Immunol 13:114-9 (2001); Matzinger P., “Tolerance, Danger, and the Extended Family,” Annu Rev Immunol 12:991-1045 (1994), which are hereby incorporated by reference in their entirety). Necrotic, but not apoptotic, cell death releases HSPs, which deliver a partial maturation signal to dendritic cells and activate the NF-κB pathway (Basu et al., “Necrotic But Not Apoptotic Cell Death Releases Heat Shock Proteins, Which Deliver a Partial Maturation Signal to Dendritic Cells and Activate the NF-kB Pathway,” Int Immunol 12:1539-46 (2000), which is hereby incorporated by reference in its entirety). HSPs released from cells may be a crucial signal that is able to activate the immune system to recognize “dangerous” physiological situations (Todryk et al., “Heat Shock Proteins Refine the Danger Theory,” Immunology 99:334-7 (2000), which is hereby incorporated by reference in its entirety). This suggests that HSPs, which are clearly intracellular proteins in all living cells, may have developed a natural extracellular function related to the early evolution of the immune response. Importantly, this would not only include HSP-peptide complexes as previously envisioned, but also HSP complexes with cellular proteins, specifically mutated, damaged, and misfolded proteins.

The concept of HSPs as danger signals is relevant to both tumor immunology and infectious agents. HSP-peptide complexes purified from tumors or from cells infected with pathogens contain tumor or pathogen-derived peptides, respectively. HSP-chaperoned peptides enter antigen presenting cells through specific receptors such as toll like receptors, scavenger receptors (LOX-1) and/or CD91, and are presented via MHC class I and II pathways, resulting in stimulation of CD8+ and CD4+ T-cells (Manjili et al., “Cancer Immunotherapy and Heat-Shock Proteins: Promises and Challenges,” Expert Opin Biol Ther 4:363-73 (2004); Delneste et al., “Involvement of LOX-1 in Dendritic Cell-Mediated Antigen Cross-Presentation,” Immunity 17:353-62 (2002), which are hereby incorporated by reference in their entirety). HSPs also induce maturation of DCs and secretion of proinflammatory cytokines. Seen in this light, HSPs confer a “danger flag” to an antigen of interest that can lead to engagement of innate pathogen recognition receptors, DC activation, and elaboration of antigen specific immunity.

The usefulness of recombinant HSP110 and GRP170 as adjuvants in cancer vaccine development has recently been demonstrated (PCT Application Publ. No. WO 01/23421; U.S. Patent Applications Publ. No. 20050202035 and 20020039583, all to Subjeck et al., which are hereby incorporated by reference in their entirety). Using newly developed purification protocols for HSP110 and GRP170, it was shown that vaccination with HSP110 or GRP170 purified from the mouse Meth A fibrosarcoma leads to complete protection (tumors initially grew, but then rapidly disappeared) (Wang et al., “Characterization of Heat Shock Protein 110 and Glucose-Regulated Protein 170 as Cancer Vaccines and the Effect of Fever-Range Hyperthermia on Vaccine Activity,” J Immunol 166:490-7(2001), which is hereby incorporated by reference in its entirety). In the murine Colon 26 tumor model, both tumor-derived HSP110 and GRP170 vaccines lead to significant growth inhibition as well as tumor specific cytotoxic T lymphocyte responses (Wang et al., “Hsp110 Over-Expression Increases the Immunogenicity of the Murine CT26 Colon Tumor,” Cancer Immunol Immunother 51:311-9 (2002), which is hereby incorporated by reference in its entirety). Since APCs are presumed to mediate this process, the activity of mouse bone marrow-derived dendritic cells (BMDCs) as vaccines was also examined following the exposure to tumor-derived HSP110 or GRP170. Mice treated with BMDCs that were pulsed with HSP110 or GRP170 purified from tumor elicited a strong anti-tumor response, which examine the mechanisms of uptake and processing of HSP110- and GRP170-protein antigen complexes by antigen presenting cells. (PCT Published Application WO 01/23421; U.S. Published Patent Applications 20050202035 and 20020039583, Subjeck et al., which are hereby incorporated by reference in their entirety).

When purified from a tumor, certain heat shock proteins (including HSP110 and GRP170) can function as effective vaccines against the same tumor. However, purification of HSP from a tumor requires a sufficient surgical specimen as a source that is often lacking and only a limited number of proteins are likely to be antigenic. Complexing HSP110 with well characterized recombinant tumor associated antigens avoids these limitations. HSP110 is a highly efficient molecular chaperone in binding to large protein substrates (Manjili et al., “Development of a Recombinant HSP110-HER-2/Neu Vaccine Using the Chaperoning Properties of HSP110,” Cancer Res 62:1737-42 (2002), which is hereby incorporated by reference in its entirety). It was demonstrated that HSP110 complexed with the intracellular domain (ICD) of Her-2/neu elicits strong antigen-specific cellular and humoral immune responses, resulting in a protective vaccine against induced mammary tumor growth. (Manjili et al., “Development of a Recombinant HSP110-HER-2/Neu Vaccine Using the Chaperoning Properties of HSP110,” Cancer Res 62:1737-42 (2002), which is hereby incorporated by reference in its entirety). Splenocytes from HSP110-ICD immunized animals elicited significant IFN-γ production upon stimulation with ICD in vitro (Manjili et al., “Development of a Recombinant HSP110-HER-2/Neu Vaccine Using the Chaperoning Properties of HSP110,” Cancer Res 62:1737-42 (2002), which is hereby incorporated by reference in its entirety). Sensitization with the HSP110-ICD complex was as effective as Complete Freund's Adjuvant (CFA) in eliciting antigen-specific IFN-γ responses in splenocytes challenged ex-vivo with ICD, as determined by ELISPOT assay. CFA is a highly potent and toxic immuno-adjuvant often used in animal studies, but not in humans. Splenocytes from mice immunized with ICD only did not show IFN-γ production upon stimulation with the ICD antigen. Vaccination with the HSP110-ICD complex induced both CD8+ and CD4+ T cell-mediated immune responses, and CD8+ T-cell activation was unaffected by CD4+ T-cell depletion (Manjili et al., “Development of a Recombinant HSP110-HER-2/Neu Vaccine Using the Chaperoning Properties of HSP110,” Cancer Res 62:1737-42 (2002), which is hereby incorporated by reference in its entirety). In a separate study, sensitization with mouse HSP110 complexed to human melanoma-associated antigen gp100 was significantly more effective in controlling tumor following challenge with B16 melanoma than sensitization with CFA and gp100 (Wang et al., “Targeted Immunotherapy Using Reconstituted Chaperone Complexes of Heat Shock Protein 110 and Melanoma-Associated Antigen gp100,” Cancer Res 63(10):2553-60 (2003), which is hereby incorporated by reference in its entirety). The HSP110/gp100 complex elicited potent antigen-specific IFN-γ production and cytotoxic T-cell responses.

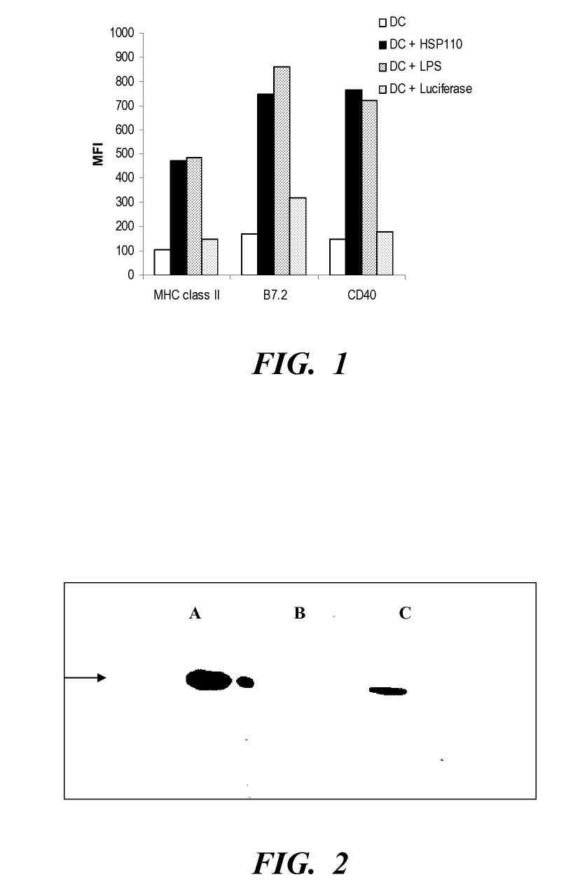

It was previously shown that mouse HSP110 in a complex with human ICD can elicit ICD-specific CD8+ T cell responses in the absence of CD4+ T cells in vivo (Manjili et al., “Development of a Recombinant HSP110-HER-2/Neu Vaccine Using the Chaperoning Properties of HSP110,” Cancer Res 62:1737-42 (2002), which is hereby incorporated by reference in its entirety). HSP110 may mature DCs, thereby bypassing the requirement for CD4+T cell help to stimulate CD8+ T cells. This hypothesis was tested by studying the interaction of recombinant HSP110 with mouse bone marrow derived DCs (BMDCs). Mouse BMDCs were prepared by the seven day culture of adherent bone marrow cells in the presence of GM-CSF (10 ng/ml). DCs (4×10⁶/ml) were cultured with mouse recombinant HSP110 (150 μg/ml), LPS (1 μg/ml), or luciferase (80-150 μg/ml) in RPMI-1640 supplemented with 10% FBS (37° C., 5% CO₂) for 20 h followed by blocking of the Fc receptors using rat anti-mouse CD16/CD32 antibody. Cells were stained with FITC-conjugated rat anti-mouse MHC class II, B7.2, and CD40 antibodies. All antibodies were used at 0.5 μg/10⁶ cells/200 μl. Cells were then subjected to flow cytometry analysis after fixing with 1% formaldehyde. Mouse serum (1 μg/200 μl) was used as isotype control and revealed <15 Mean Fluorescence Intensity (MFI). As shown in FIG. 1, HSP110 induced BMDCs to up-regulate surface expression of MHC class II, B7.2, CD40 molecules, confirming the hypothesis that HSP110 treatment matures dendritic cells.

As shown in Table 2 below, HSP110 also induced mouse BMDCs to secrete proinflammatory cytokines, IL-6, IL-12, and TNF-α. Mouse BMDCs (4×10⁶/ml) were cultured with recombinant HSP110 (150 μg/ml), LPS (1 μg/ml), or luciferase (80-150 μg/ml) in RPMI-1640 (37° C., 5% CO₂) for 20 h. Supernatants were collected and subjected to flow cytometry-based analysis using the cytokine specific antibody-coated beads (R & D System, Inc., Minneapolis, Minn.). Appropriate controls and standards were used. HSP110 endotoxin level was below the limit of detection (lower than 20 EU/mg).

TABLE 2
Secretion of Pre-Inflammatory Cytokines by Mouse BMDC

| DC treatment | IL-6 (pg/ml) | IL-12 (pg/ml) | TNF-α (pg/ml) |
|---|---|---|---|
| Control DCs | 119 | 0 | 0 |
| HSP110 | 49038 | 883 | 1919 |
| LPS | 36149 | 143 | 1371 |
| Luciferase | 540 | 0 | 10 |

### Example 2

### Generation of HSP110/Asp f2 Complex

Asp f2 was selected as the initial fungal antigen for evaluation because it has been the most extensively characterized in human and mouse models of allergic bronchopulmonary aspergillosis (ABPA) (Banerjee et al., “Purification of a Major Allergen, Asp f2 Binding to IgE in Allergic Bronchopulmonary Aspergillosis, From Culture Filtrate of Aspergillus Fumigatus,” J Allergy Clin Immunol 99:821-7 (1997); Banerjee et al., “Molecular Cloning and Expression of a Recombinant Aspergillus Fumigatus Protein Asp fII With Significant Immunoglobulin E Reactivity in Allergic Bronchopulmonary Aspergillosis,” J Lab Clin Med 127:253-62 (1996); Banerjee et al., “Immunological Characterization of Asp f2, a Major Allergen From Aspergillus Fumigatus Associated With Allergic Bronchopulmonary Aspergillosis,” Infect Immun 66:5175-82 (1998); Banerjee et al., “Conformational and Linear B-Cell Epitopes of Asp f2, a Major Allergen of Aspergillus Fumigatus, Bind Differently to Immunoglobulin E Antibody in the Sera of Allergic Bronchopulmonary Aspergillosis Patients,” Infect Immun 67:2284-91 (1999), which are hereby incorporated by reference in their entirety). It is expressed as a major cell-associated protein within 24 h of in vitro fungal culture, comprising 20 to 40% of total hyphal protein (Banerjee et al., “Immunological Characterization of Asp f2, a Major Allergen From Aspergillus Fumigatus Associated With Allergic Bronchopulmonary Aspergillosis,” Infect Immun 66:5175-82(1998), which is hereby incorporated by reference in its entirety). It binds to laminin in vitro, suggesting that it is expressed on the cell surface and has a role in interacting with host extracellular matrix proteins (Banerjee et al., “Immunological Characterization of Asp f2, a Major Allergen From Aspergillus Fumigatus Associated With Allergic Bronchopulmonary Aspergillosis,” Infect Immun 66:5175-82 (1998), which is hereby incorporated by reference in its entirety). Thus, during infection with A. fumigatus, it is expected that Asp f2 will be expressed in large amounts relative to other proteins and will be recognized by host defense cells in mice previously sensitized with this antigen. Specific T-cell epitopes of Asp f2 have been identified in mice (Svirshchevskaya et al., “Immune Response Modulation by Recombinant Peptides Expressed in Virus-Like Particles,” Clin Exp Immunol 127:199-205 (2002), which is hereby incorporated by reference in its entirety) and in patients with ABPA (Rathore et al., “T Cell Proliferation and Cytokine Secretion to T Cell Epitopes of Asp F2 in ABPA Patients,” Clin Immunol 100:228-35 (2001), which is hereby incorporated by reference in its entirety). Asp f2 peptide-based immunotherapy has been used successfully in the modulation of the Asp f2-induced immune response in mice (Svirshchevskaya et al., “Immune response Modulation by Recombinant Peptides Expressed in Virus-Like Particles,” Clin Exp Immunol 127:199-205 (2002), which is hereby incorporated by reference in its entirety), lending support to the concept of using this antigen in immune-based strategies. Sensitization with HSP110/Asp f2 complex is hypothesized to elicit type 1 cytokine responses, whereas sensitization with Asp f2 alone will elicit predominantly type 2 responses. It is also expected that sensitization with HSP110/Asp f2 complex will be protective following subsequent intratracheal challenge with Aspergillus.

The non-covalent binding of the HSP110 to Asp f2 at 43° C. was carried essentially as previously described for HSP110 antigen binding (Manjili et al., “Development of a Recombinant HSP110-HER-2/Neu Vaccine Using the Chaperoning Properties of HSP110,” Cancer Res 62:1737-42 (2002), which is hereby incorporated by reference in its entirety). Briefly, HSP110 and Asp f2 were incubated at 1:1 molar ratio at 43° C. for 1 hour. The HSP110-Asp f2 complex was incubated with HSP110 antiserum (1:200) or an irrelevant antibody (rabbit antiserum) as a specificity control and run over a protein A-Sepharose column. The column was washed to remove non-specific binding. When heated to 43° C., HSP110 molecules form non-covalent complexes with each other and with antigens, yielding bands of unpredictable molecular weight. For this reason, the HSP110 complexes were dissociated using 2× SDS sample buffer and boiled for 5 minutes. The eluate containing dissociated HSP110 and Asp f2 molecules were then subjected to Western blot using monoclonal anti-Asp f2 antibody. As shown in FIG. 2, HSP110 binds to Asp f2 under heat shock conditions.

### Example 3

### Immunogenicity of HSP/Asp f2 Complex

To further characterize the effect of HSP110 and the HSP/Asp f2 complex on DC activation, CD86 expression was evaluated. T cell activation is dependent upon signals delivered through the antigen-specific T cell receptor and accessory receptors on the T cell. A primary costimulatory signal is delivered through the CD28 receptor after engagement of its ligands, CD80 (B7.1) or CD86 (B7.2). Integration of signals through this family of costimulatory receptors and their ligands is critical for IL-2-dependent activation of T-cell responses. Bone marrow was harvested from C.C3-TLR 4^{Lps-d}/J and control BALB/cJ mice (Jackson Laboratories, Bar Harbor, Me.). Red blood cells were lysed and remaining cells were incubated with 50 ng GM-CSF/ml complete RPMI and pulsed with the same on days 2 and 5. On day six, 10⁶ cells/ml complete RPMI were plated in each well of a 6 well plate and pulsed and incubated overnight with 150 μg HSP110, 50 μg Asp f2, HSP110/Asp f2 protein complex, LPS (1 μg/ml), or vehicle. The cells were harvested on day seven and analyzed for CD86 expression by flow cytometry.

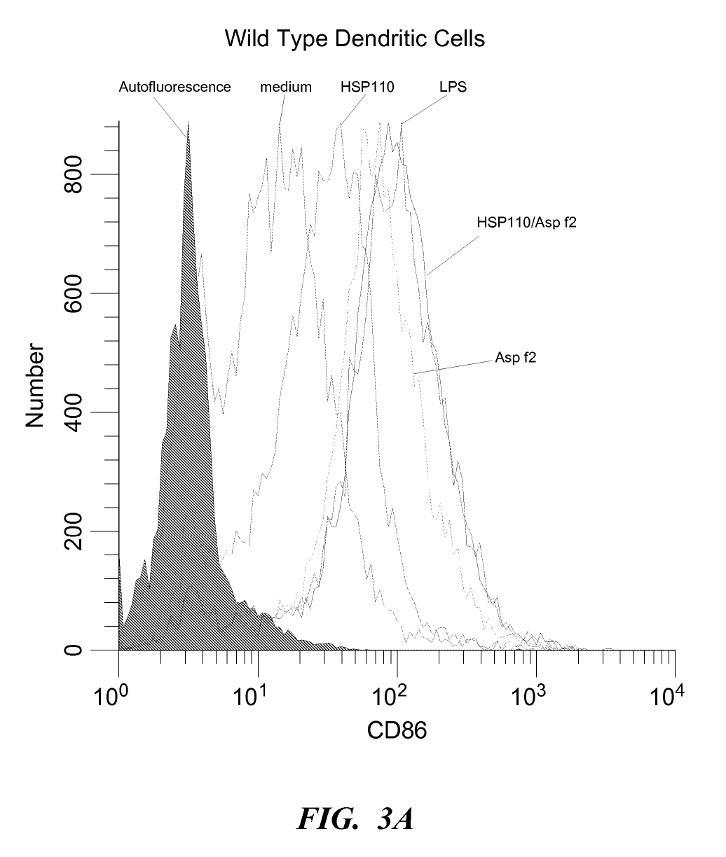

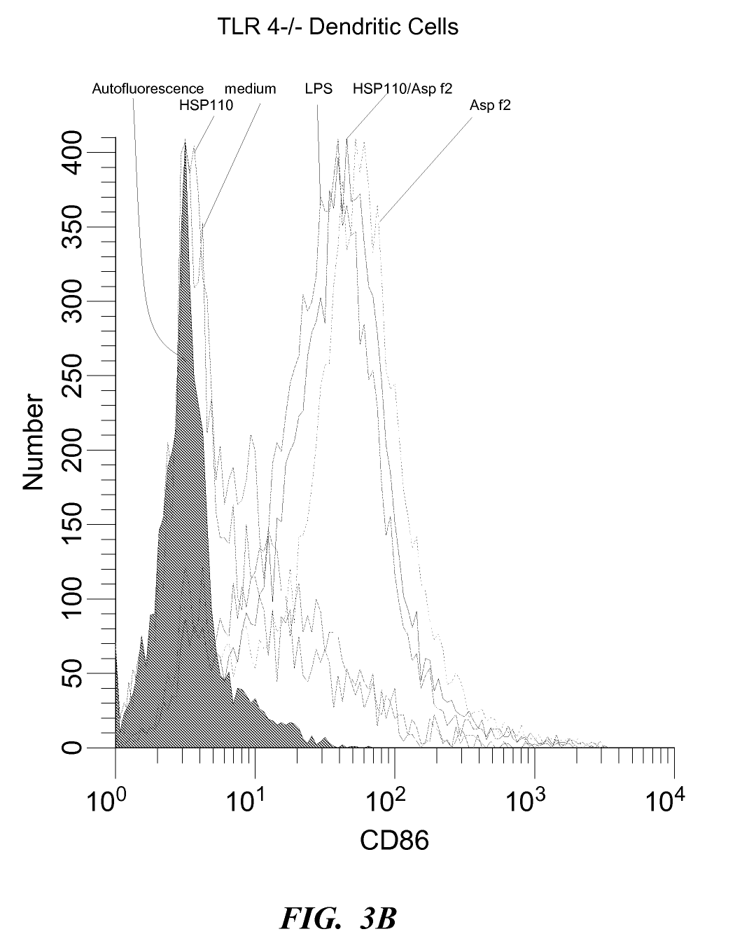

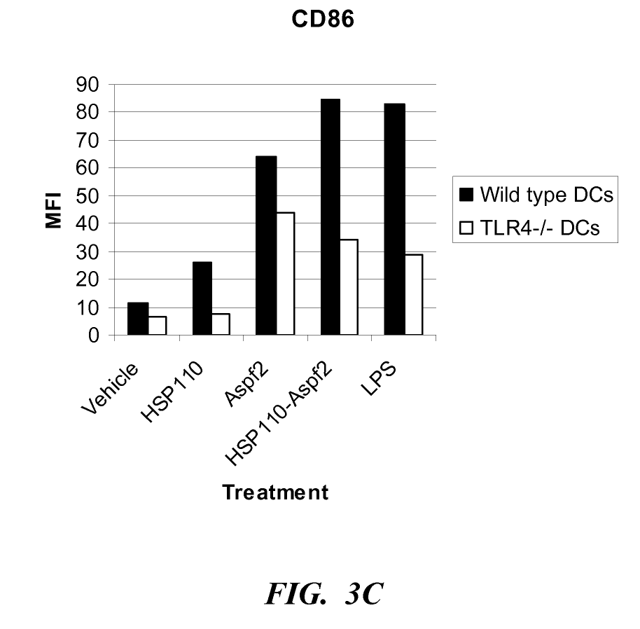

HSP110 increased CD86 surface expression in wildtype DCs compared to unstimulated cells, as shown in FIG. 3A. In contrast, no increase in CD86 expression occurred in TLR 4−/−DCs after HSP110 stimulation (FIG. 3B), indicating that HSP110-mediated stimulation of CD86 expression is TLR 4-dependent. Asp f2 alone was a more potent stimulus of CD86 expression than HSP110 in wildtype DCs, and the HSP110/Asp f2 complex was comparable to Asp f2 alone in its ability to increase stimulation of CD86 expression (FIG. 3A). In TLR 4−/−DCs, Asp f2 stimulated CD86 expression to a similar degree as in wildtype DCs. Asp f2 alone and complexed to HSP110 generated similar levels of CD86 expression in TLR 4−/−DCs. Mean fluorescent intensities of CD86 expression are summarized in FIG. 3C. These data indicate that HSP110 augments CD86 expression through a TLR-4-dependent pathway, whereas Asp f2 induces CD86 expression largely independently of TLR 4.

These results provide the rationale to characterize DC responses to HSP110/Asp2 sensitization in vivo and the role of TLR 4 signaling in mediating immunogenicity in vivo.

### Example 4

### Immunogenicity of HSP110/Asp f2 In Vivo

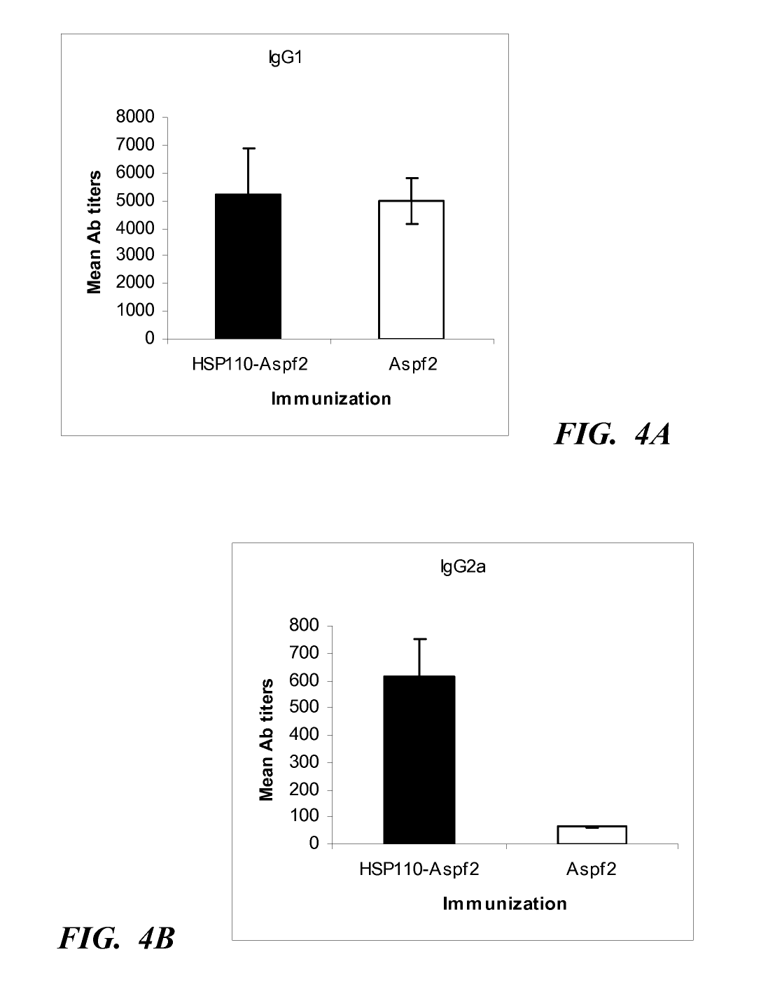

C57BL/6 mice (3 per group) were sensitized on days 0 and 14 with i.p. HSP110/Asp f2 complex (10 g Asp f2+25 μg Hsp110 per mouse, corresponding to 1:1 molar ratio) and the appropriate controls, Asp f2 alone (10 μg/mouse), HSP110 alone (25 μg/mouse), or vehicle were administered. Results are shown in FIGS. 4A-B. On day 14, antibody levels were at background levels in all groups. On day 25, serum IgG1 levels specific for Asp f2 were similar in mice immunized with the HSP110/Aspf2 complex and Asp f2 alone, as shown in FIG. 4A. In contrast, serum Asp f2-specific IgG2a levels were significantly higher in HSP110/Asp f2 compared to Asp f2 alone recipients (617±137 vs. 63±3 units respectively; p=0.005), as shown in FIG. 4B. Titers in mice receiving HSP110 alone and vehicle were close to nil.

The global antibody response may include protective, non-protective, and deleterious antibodies. Immunoglobulin class switching is under the regulatory control of T-cell cytokine responses. IL-4 primes B lymphocytes to switch to IgG1 (Snapper et al., “Interferon-Gamma and B Cell Stimulatory Factor-1 Reciprocally Regulate Ig Isotype Production,” Science 236(4804):944-7 (1987); Snapper et al., “B Cell Stimulatory Factor-1 (Interleukin 4) Prepares Resting Murine B Cells to Secrete IgG1 Upon Subsequent Stimulation With Bacterial Lipopolysaccharide,” J Immunol 139:10-7 (1987), which are herebyincorporated by reference in their entirety). The regulation of switching to production of IgG2a is the reciprocal of IgG 1; IgG2a responses are induced by IFN-γ and suppressed by IL-4 (Snapper et al., “Interferon-Gamma and B Cell Stimulatory Factor-1 Reciprocally Regulate Ig Isotype Production,” Science 236(4804):944-7 (1987), which is hereby incorporated by reference in its entirety). An increase in antigen-specific IgG2a responses reflects endogenous IFN-γ production. Thus, an increase in antigen-specific IgG2a responses in these studies likely reflects skewing of T-cell responses to the type I phenotype. It is also possible that HSP110 directly activates NK cells, which are also a source of IFN-γ production. Asp f2-specific T-cell responses will be characterized at the single cell level using the ELISPOT.

### Example 5

### Single Cell-Cytokine Analysis

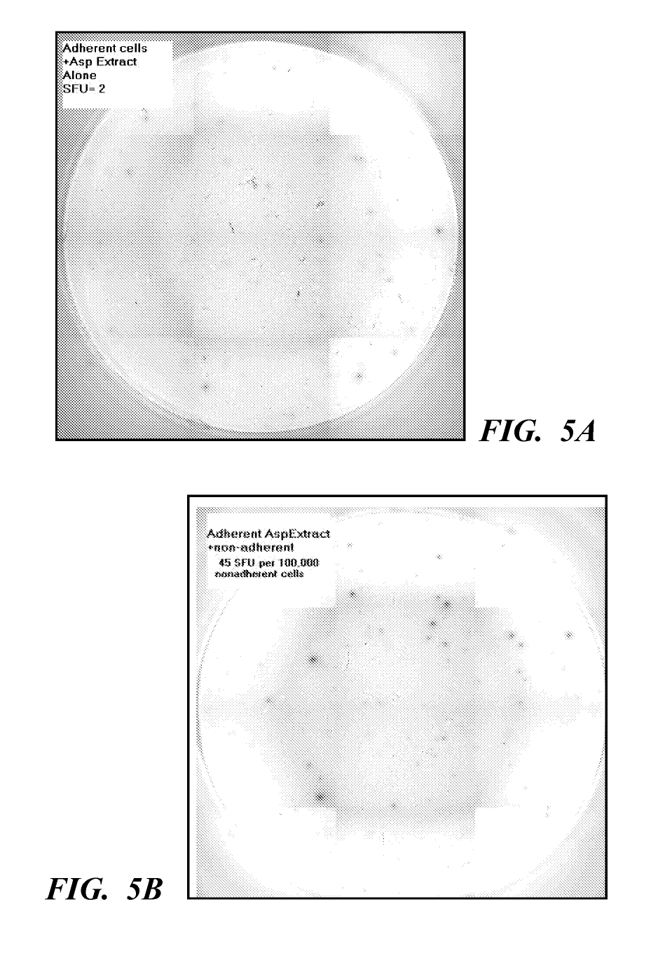

To evaluate the immunogenicity of vaccines aimed at augmenting T-cell immunity, characterization of cytokine responses at the single cell level is useful because antigen presentation and development of T-cell phenotypes occur precisely at the level of cell-cell interactions. The ELISPOT assay uses an antibody-based technique for quantitation of single cells secreting cytokines (spot forming units) in response to stimulation. The ELISPOT assay is highly reproducible and sufficiently sensitive to detect 1 cytokine secreting T-cell among 100,000 (Asai et al., “Evaluation of the Modified ELISPOT Assay for Gamma Interferon Production in Monitoring of Cancer Patients Receiving Antitumor Vaccines,” Clin Diagn Lab Immunol 7:145-54 (2000), which is hereby incorporated by reference in its entirety). The ELISPOT assay will enable the determination of the proportion of T-cells producing IFN-γ and IL-4 in response to ex vivo stimulation with Aspergillus antigens. An illustrative ELISPOT is shown in FIGS. 5A-B. Adherent cells were stimulated overnight with A. fumigatus extract (10 ug/ml), followed by ELISPOT detection without addition of non-adherent cells (which contain the lymphocyte responder population). As shown in FIG. 5A, there were virtually no spot forming units detected. In contrast, when the non-adherent cell fraction was added, 45 spot forming units (SFU)/100,000 cells (corresponding to IFN-γ positive cells) were observed, as shown in FIG. 5B.

### Example 6

### Characterization of Aspergillus Infection in Immunocompromised Mouse Models

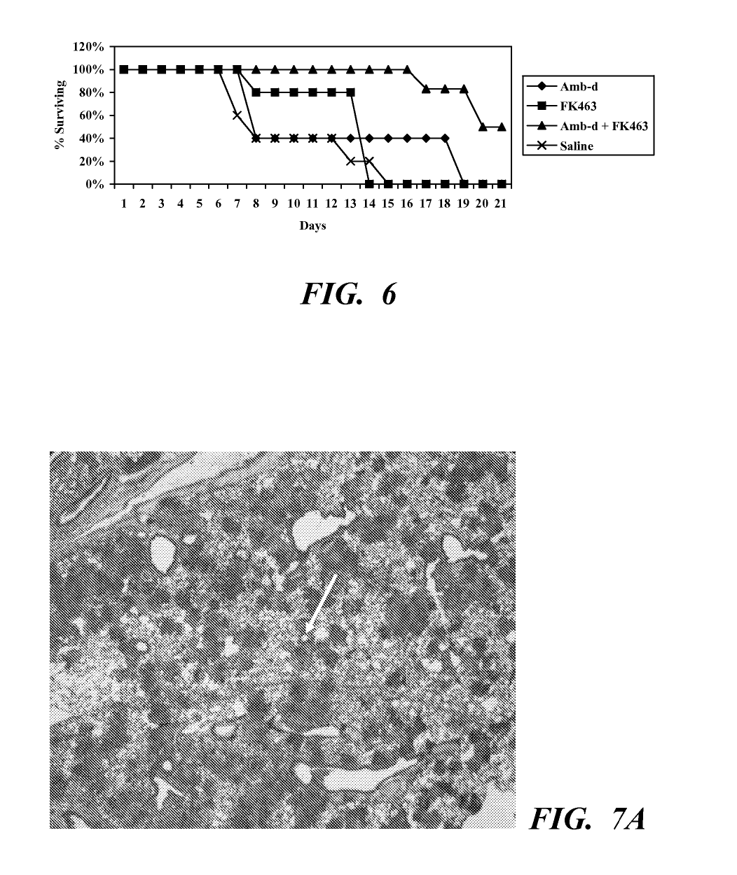

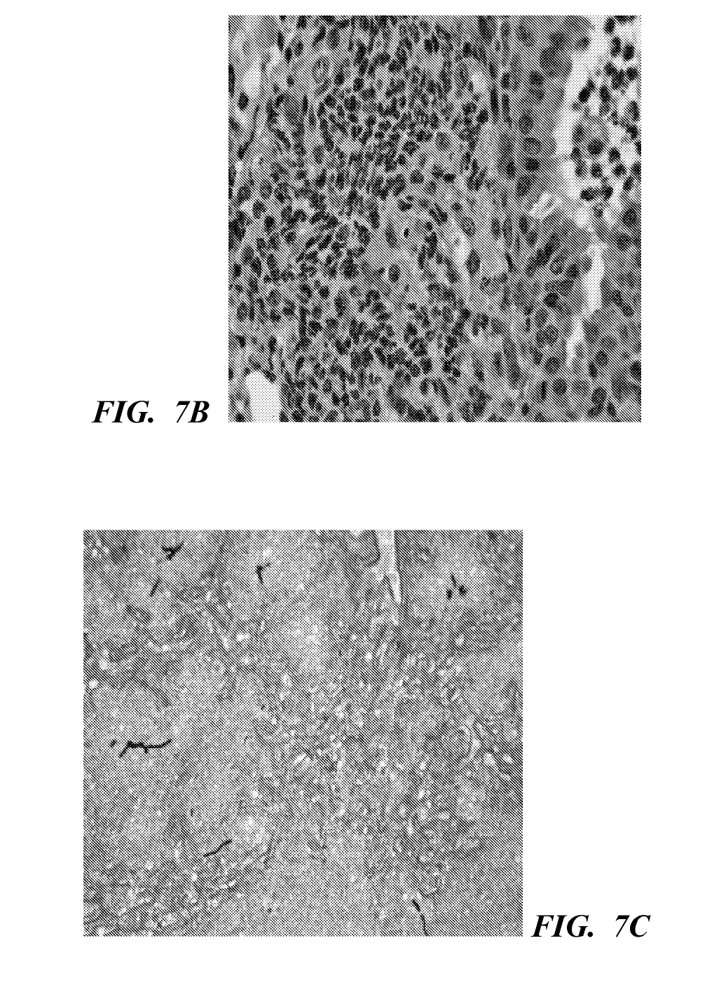

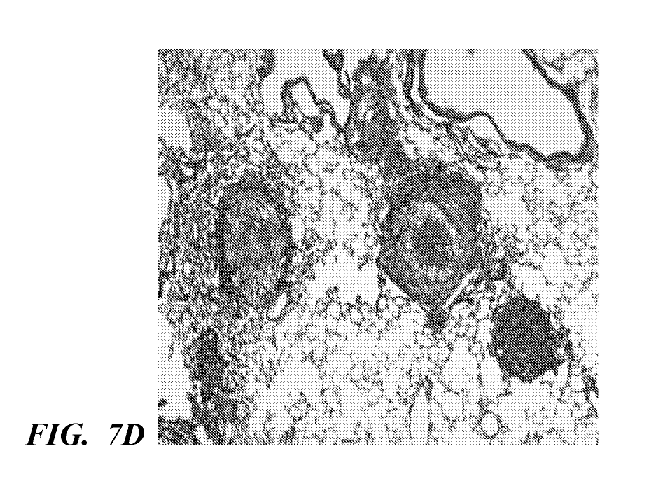

The p47^{Phox-/-}mouse model of chronic granulomatous disease (CGD) and corticosteroid-treated mice were used in experiments that characterized the role of fungal catalase genes in the virulence of Aspergillus nidulans (Chang et al., “Virulence of Catalase-Deficient Aspergillus Nidulans in p47(Phox)−/−Mice. Implications for Fungal Pathogenicity and Host Defense in Chronic Granulomatous Disease,” J Clin Invest 101:1843-50 (1998), which is hereby incorporated by reference in its entirety). CGD is an inherited disorder of the NADPH oxidase that is characterized by recurrent bacterial and fungal infections and abnormally exuberant inflammatory responses, such as granulomatous enteritis and genitourinary obstruction. Invasive aspergillosis is the most important cause of mortality in CGD (Cohen et al., “Fungal Infection in Chronic Granulomatous Disease. The Importance of the Phagocyte in Defense Against Fungi,” Am J Med 71:59-66 (1981); Segal et al., “Aspergillus Nidulans Infection in Chronic Granulomatous Disease,” Medicine (Baltimore) 77:345-54 (1998); Segal et al., “Invasive Aspergillosis in Chronic Granulomatous Disease,” The Aspergillus website (2003); Winkelstein et al., “Chronic Granulomatous Disease: Report on a National Registry of 368 Patients,” Medicine 79:153-69 (2000), which are hereby incorporated by reference in their entirety). Both the p47^{Phox-/-}(Chang et al., “Virulence of Catalase-Deficient Aspergillus Nidulans in p47(Phox)−/−Mice. Implications for Fungal Pathogenicity and Host Defense in Chronic Granulomatous Disease,” J Clin Invest 101:1843-50 (1998), which is hereby incorporated by reference in its entirety) and X-linked (Pollock et al., “Mouse Model of X-Linked Chronic Granulomatous Disease, an Inherited Defect in Phagocyte Superoxide Production,” Nat Genet 9:202-9 (1995), which is hereby incorporated by reference in its entirety) CGD knockout mice are highly susceptible to experimental pulmonary Aspergillus infection. CGD mice were subjected to Aspergillus infection to evaluate combination antifungal regimens. A morbidity-free survival experiment evaluating combination amphotericin B (Amb-d; 1 mg/kg daily for 5 days) plus the echinocandin FK463 (micafungin; 10 mg/kg daily for 5 days) versus monotherapy following intratracheal Aspergillus challenge (1.25×10⁴ CFU/mouse) is shown in FIG. 6. The histopathology of early and late aspergillosis in CGD mice was also characterized, as shown in FIGS. 7A-D. On day 4 after intratracheal challenge with 1.25×10³ CFU/mouse, multiple discrete foci of inflammation were present in the lung (H&E 50×), as shown in FIG. 7A. Higher power magnification (63033 ) showing neutrophils surrounding a hyphal element (arrow), as shown in FIG. 7B. GMS stain shows invasive hyphae (200×), shown in FIG. 7C. At 9 weeks after sub-lethal challenge, well-defined granulomata persist (H&E 200×) and hyphae were occasionally observed, as shown in FIG. 7D.

### Example 7

### HSP110/Asp f2 Increase of Innate and Antigen-Specific Immunity In Vivo

To test the belief that TLR 4 activation is a requisite for immunogenicity of the HSP110/Asp f2 complex in vivo, in vivo studies on DC activation will be carried out.

Vaccination protocol. Wildtype and TLR 4−/−mice (5 mice/group; 6-8 weeks of age) will be sensitized on days 0 and 14 with i.p. HSP110/Asp f2 complex (10 μg Asp f2+25 g Hsp110 per mouse corresponding to 1:1 molar ratio) and the appropriate controls: vehicle, Asp f2 alone (10 μg/mouse), HSP110 alone (25 μg/mouse), or Asp f2 (10 μg/mouse) together with CpG oligodeoxynucleotide sequences (50 μg/mouse). CpG sequences stimulate TLR 9 signaling and are expected to induce DC activation and type I antigen-specific immunity. CpG sequences as adjuvants have been effective in both experimental invasive (Bozza et al., “Vaccination of Mice Against Invasive Aspergillosis With Recombinant Aspergillus Proteins and CpG Oligodeoxynucleotides as Adjuvants,” Microbes Infect 4:1281-90 (2002), which is hereby incorporated by reference in its entirety) and allergic (Banerjee et al., “Modulation of Airway Inflammation by Immunostimulatory CpG Oligodeoxynucleotides in a Murine Model of Allergic Aspergillosis,” Infect Immun 72(10):6087-94 (2004), which is hereby incorporated by reference in its entirety) aspergillosis, and are thus highly acceptable standards by which to evaluate HSP110/Asp f2 as a candidate vaccine. The effect of HSP110/Asp f2 and appropriate controls in vivo on both innate and antigen specific responses will be characterized. These studies will comprehensively link innate and antigen-specific functions induced by HSP110/Asp f2 sensitization, and will characterize the role of TLR 4 signaling.

Groups of mice (5 per genotype per time point) will be sacrificed on days 15 and 25 after the initial sensitization DC activation in vivo and antigen-dependent immunity will be determined as described below.

DC activation. Pulmonary DCs transport the conidia and hyphae of A. fumigatus from the airways to the draining lymph nodes and initiate T helper responses to the fungus (Bozza et al., “Dendritic Cells Transport Conidia and Hyphae of Aspergillus Fumigatus From the Airways to the Draining Lymph Nodes and Initiate Disparate Th Responses to the Fungus,” J Immunol 168: 1362-71 (2002), which is hereby incorporated by reference in its entirety). TLR pathways (principally TLR 2 and 4) initiate and coordinate inflammatory responses to Aspergillus infection. Because of the key role of DCs in initiating and regulating immunity to Aspergillus infection, the effect of HSP110/Asp f2 sensitization on pulmonary and splenic DCs and the role of TLR 4 signaling in mediating DC activation will be characterized. Whole lungs will be recovered and tissue will be physically disrupted using a metal sieve. Cell strainers (BD Falcon; BD Biosciences Discovery Labware, San Jose, Calif.) 100 μm in diameter will be used to remove debris. The percentage yield and total number of CD11c⁺ cells and the expression of CD86 and MHC II on this cell population will be quantified using previously described methods (Piggott et al., “MyD88-Dependent Induction of Allergic Th2 Responses to Intranasal Antigen,” J Clin Invest 115(2):459-67 (2005), which is hereby incorporated by reference in its entirety). Single cell splenocyte suspensions will be generated by mechanical disruption through a strainer, and surface expression of CD86 and MHC II will be determined. Based on prior in vitro studies of DC activation, it is believed that sensitization with HSP110 alone and complexed to Asp f2 will activate pulmonary and splenic DCs in wildtype mice, and that activation will be attenuated in TLR 4−/−mice. The effect of sensitization with HSP110/Asp f2 and appropriate controls on TLR 4 expression on pulmonary and splenic DCs from wildtype mice will also be assessed. Since IFN-γ augments TLR 4 (and TLR 2) expression on a variety of antigen presenting cells (as describe in the Background section) sensitization with HSP110 alone and complexed to Asp f2 may increase TLR 4 expression on DCs by stimulating production of IFN-γ.

Antigen-dependent immunity. Whether sensitization with HSP110/Asp f2 will lead to more robust Asp f2-specific type 1 T-cell immunity in wildtype compared to TLR 4−/−mice will be tested. As shown in FIG. 4B, HSP110/Asp f2 sensitization induced a 10-fold increase in Asp f2-specific IgG2a compared to sensitization with Asp f2 alone in wildtype mice. Augmentation of IgG2a is a reflection of IFN-γ production. It is believed that the increase in Asp f2-specific IgG2a results from skewing of T-cell responses to the type I phenotype. However, it is possible that HSP110 directly activated NK cells, which are also a source of IFN-γ production. Therefore, Asp f2-specific T-cell responses at the single cell level will be characterized using the ELISPOT assay.

Single cell lymph node and splenocyte suspensions, which contain both APCs and responder cells, will be generated by mechanical separation through a 100 um nylon filter. Because IFN-γ is generated by both CD4+ and CD8+ cells, enriched CD4+ and CD8+ fractions will be generated by depletion using anti-CD4+ and anti-CD8+ Miltenyi magnetic beads and columns (Miltenyi Biotech, Inc., Auburn, Calif.). As a control for specificity of responder cells, fractions depleted of both CD4+ and CD8+ T-cells will be used in parallel. Splenocytes (5×10⁵/well) will be incubated with Asp f2 (10 μg/ml), an irrelevant control recombinant protein (HER-2/neu, 10 μg/ml), con A (5 μg/ml) or HSP110 (10 μg/ml) in complete medium at 37° C. in an atmosphere of 5% CO₂ for 20-24 h. IFN-γ or IL-4 spots are detectable using capture antibody (10 μg/ml anti-mouse IFN-γ or IL-4 antibodies (Pharmingen, San Diego, Calif.), detection antibody (5 μg/ml biotinylated IFN-γ or IL-4 antibodies (Pharmingen, San Diego, Calif.), 0.2 U/ml alkaline phosphatase avidin D (Vector Laboratories, Burlingame, Calif.), and 50 μl BCIP/NBT solution (Boehringer Mannheim, Indianapolis, Ind.). Spots can be counted by a Carl Zeiss Vision ELISPOT reader (Cell Technology, Inc., Columbia, Md.), and the readout is spot forming units per 100,000 cells. The specificity of the HSP110/Asp f2 complex will be demonstrated using the ELISPOT method based on IFN-γ+ T-cells that are significantly greater following ex vivo challenge with Asp f2 compared with an irrelevant antigen (HER-2/neu). Type 1 antigen-specific T-cell responses will be defined by the proportion of IFN-γ positive cells and the ratio of IFN-γ/IL-4 producing cells following ex vivo stimulation with Asp f2.

### Example 8

### HSP110 +/−Asp f2 Activation of DCs from Immunocompromised Mice

The in vitro results presented above (showing DC activation by HSP110 alone and complexed to Asp f2) will be correlated with activation in vivo following vaccination and Aspergillus challenge.

DC activation in vitro. HSP110-mediated activation will be attenuated in bone marrow-derived DCs from CGD mice and corticosteroid-treated wildtype DCs. CGD mice and corticosteroid treated mice are both susceptible to experimental aspergillosis. The effect of HSP110 on activation of DCs from CGD mice and corticosteroid-treated DCs will be characterized. Parallel experiments will be conducted on the following bone marrow derived DC preparations: 1) wildtype DCs; 2) wildtype DCs plus dexamethasone (DEX 10⁻⁹M to 10⁻⁶M); and 3) DCs derived from CGD mice. Bone marrow-derived DCs will be generated and stimulated with HSP110/Asp f2 and appropriate controls (vehicle, HSP110 alone, Asp f2 alone, or LPS) as described in Example 1 above. DC activation will be characterized by CD86 and MHC II expression, and TLR 4 expression will be quantified.

The role of HSP110 on activation of DCs from CGD mice is difficult to predict, because CGD results from a defect in the NADPH oxidase. NADPH oxidase-derived reactive oxidants not only play a role in host defense but also in cell signaling. It has been shown that NADPH oxidase-derived reactive oxidants are required for Kupffer cell NF-κB activation following challenge with the peroxisomal proliferators (Rusyn et al., “Oxidants From Nicotinamide Adenine Dinucleotide Phosphate Oxidase are Involved in Triggering Cell Proliferation in the Liver Due to Peroxisome Proliferators,” Cancer Res 60:4798-80 (2000), which is hereby incorporated by reference in its entirety) and ethanol (Kono et al., “NADPH Oxidase-Derived Free Radicals Are Key Oxidants in Alcohol-Induced Liver Disease,” J Clin Invest 106:867-72 (2000), which is hereby incorporated by reference in its entirety). Pulmonary NF-κB activation was also attenuated in CGD mice following intraperitoneal and aerosolized challenge with endotoxin (Koay et al., “Impaired Pulmonary NF-κB Activation in Response to Lipopolysaccharide in NADPH Oxidase-Deficient Mice,” Infect Immun 69:5991-6 (2001), which is hereby incorporated by reference in its entirety). The interaction of NADPH oxidase-derived reactive oxidants and nitric oxide (NO) in T-cell responses has also been explored. Activated peritoneal macrophages from CGD mice elicited reduced T-cell proliferative responses following presentation of a peptide immunogen known to elicit autoimmune encephalomyelitis in vivo; a normal proliferative response was restored upon addition of an iNOS inhibitor (van der Veen et al., “Superoxide Prevents Nitric Oxide-Mediated Suppression of Helper T Lymphocytes: Decreased Autoimmune Encephalomyelitis in Nicotinamide Adenine Dinucleotide Phosphate Oxidase Knockout Mice,” J Immunol 164:5177-83 (2000), which is hereby incorporated by reference in its entirety). Consistent with the in vitro findings, CGD mice were protected from autoimmune encephalomyelitis following in vivo challenge with this peptide. These studies illustrate the broad importance of reactive oxidants as cell signaling agents regulating inflammatory responses. An attenuated response in DCs from CGD mice to HSP110 would argue that HSP110 activation of DCs is, at least in part, dependent on a functional NADPH oxidase. This finding would have important implications on immunotherapies aimed at DC activation and the role of reactive oxidant signaling in DC maturation. Dexamethasone inhibits DC maturation in vitro (Matsue et al., “Contrasting Impacts of Immunosuppressive Agents (Rapamycin, FK506, Cyclosporin A, and Dexamethasone) on Didirectional Dendritic Cell-T Cell Interaction During Antigen Presentation,” J Immunol 169(7):3555-64 (2002); Matasic et al., “Dexamethasone Inhibits Dendritic Cell Maturation by Redirecting Differentiation of a Subset of Cells,” which are hereby incorporated by reference in their entirety). It is therefore expected that HSP110 alone and the HSP110/Asp f2 complex will stimulate maturation of dexamethasone-treated DCs, but that the response will be attenuated compared to DCs not exposed to steroids.

### Example 9

### DC Activation In Vivo in CGD and Corticosteroid Mice

HSP110 alone and complexed to Asp f2 will augment DC activation in immunocompromised mice, but the activation may be attenuated compared to immunocompetent wildtype mice. CGD and corticosteroid-treated wildtype mice (5 per genotype per time point) will be immunized with HSP110/Asp f2 or appropriate control antigen as described above (vaccination protocol) followed by challenge with either a sub-lethal inoculum (10% of the LD50 inoculum) of A. fumigatus or sham infection with vehicle. Mice will be sacrificed on days 7 and 21 after Aspergillus challenge. Activation of pulmonary and splenic DCs based on surface expression of CD80 and MHC II and quantitation of TLR 4 will be performed as described above (DC activation). These results will be correlated with in vitro stimulation studies performed on bone marrow-derived DCs from CGD mice and corticosteroid-treated wildtype DCs.

A key goal of the present invention is to understand the biology of HSP110/Asp f2 and to correlate its mechanisms of action with protection in experimental aspergillosis, in particular, the generation of an important foundation related to the mechanisms by which HSP110 alone and complexed to Asp f2 activates DCs and antigen specific immunity. As shown in FIGS. 3A-C, HSP110 activates DCs through TLR 4 signaling and HSP110/Asp f2 augments Asp f2-specific IgG2a responses in mice (a reflection of endogenous IFN-γ production), as shown in FIG. 4B. These results will be built upon by characterizing the role of TLR 4 signaling on HSP110/Asp f2-mediated DC activation and antigen specific immunity in vivo, and the ability of HSP110 alone and complexed to Asp f2 to activate DCs in CGD and corticosteroid-treated mice. It is believed that DC activation in vivo will be predictive of type I cellular immunity and protection against Aspergillus challenge in immunocompromised mice.

### Example 10

### Protection Conferred by HSP110/Asp F2 in Experimental Aspergillosis in Immunocompromised Mice

This experiment will test the belief that sensitization with HSP110/Asp f2 will confer protection against subsequent Aspergillus challenge in immunocompromised mice. The principal endpoint for protection will be morbidity requiring euthanasia. Protection in the p47^{phox-/-}mouse model of chronic granulomatous disease and in corticosteroid-treated wildtype mice will be evaluated.

Immunocompromised mice (either a knockout model or pharmacologic immunosuppression) are required because unmanipulated C57BL/6 mice are resistant to intratracheal challenge with A. fumigatus. A limitation of these models is that they do not perfectly reflect the human condition and each has its own strengths and weaknesses. For this reason, two groups of immunocompromised mice will be selected that model distinct human conditions. If results from these studies are promising, the benefit of vaccination in other mouse models of invasive aspergillosis, such as neutropenic mice and stem cell transplant recipients, will also be evaluated.

Rationale for chronic granulomatous disease mice. CGD mice have been selected for the following reasons. Firstly, CGD results from a well-defined defect in the NADPH oxidase, whereas lymphocyte function appears to be intact in CGD mice. Therefore a vaccine candidate is more likely to be immunogenic in CGD than in other immunocompromised mice. However, possibility that NADPH oxidase-derived reactive oxidants may have an important signaling role in T-cell responses is also considered, and consequently, dysregulation of T-cell responses may occur in CGD. Second, CGD mice do not require exogenous immunosuppressive agents to render them susceptible to infection. Furthermore, CGD mice have a low rate of spontaneous infections compared to other immunocompromised mouse models in which spontaneous bacterial infections can confound results. Finally, CGD mice develop chronic Aspergillus infection that persists for at least 9 weeks after challenge with sublethal inocula of Aspergillus (as shown in FIG. 7D). Thus, CGD mice are an ideal model of chronic aspergillosis in which fungal burden, pathology, and cytokine responses can be evaluated over prolonged periods.

Given that CGD results from a primary phagocytic disorder, it appears that there is a justifiable rationale to pursuing a vaccine-based strategy that augments cellular (acquired) immunity. As described in the Background section above, the immunologic response to Aspergillus infection is under complex regulatory control. Whereas alveolar macrophages and neutrophils are the key effector cells driving innate immunity, the overall inflammatory response is modulated by antigen presenting cells via TLR pathways and T-cells that mediate the cytokine response. There is precedent that IFN-γ augments host defense against Aspergillus in CGD patients. It has been shown that administration of IFN-γ to CGD patients augmented the in vitro ability of CGD neutrophils to damage Aspergillus hyphae (Rex et al., “In Vivo Interferon-Gamma Therapy Augments the in Vitro Ability of Chronic Granulomatous Disease Neutrophils to Damage Aspergillus Hyphae,” J Infect Dis 163:849-52 (1991), which is hereby incorporated by reference in its entirety). In a multicenter, randomized, double blinded, placebo-controlled study, prophylactic IFN-γ reduced the number of serious infections by over 70% (“A Controlled Trial of Interferon Gamma to Prevent Infection in Chronic Granulomatous Disease,” The International Chronic Granulomatous Disease Cooperative Study Group, N Engl J Med 324:509-16 (1991), which is hereby incorporated by reference in its entirety). In contrast to earlier studies, no significant differences occurred between the IFN-γ and placebo groups with regard to reactive oxidant generation. Subsequent studies in CGD patients (Woodman et al., “Prolonged Recombinant Interferon-Gamma Therapy in Chronic Granulomatous Disease: Evidence Against Enhanced Neutrophil Oxidase Activity,” Blood 79:1558-62 (1992); Muhlebach et al., “Treatment of Patients With Chronic Granulomatous Disease With Recombinant Human Interferon-Gamma Does Not Improve Neutrophil Oxidative Metabolism, Cytochrome b558 Content or Levels of Four Anti-Microbial Proteins,” Clin Exp Immunol 88:203-6 (1992), which are hereby incorporated by reference in their entirety) confirmed that IFN-γ did not improve NADPH oxidase function or increase levels of its constituent proteins. Thus, the benefit of IFN-γ prophylaxis in CGD likely occurs through augmentation of oxidant-independent antimicrobial pathways. Seen in this light, HSP110/Asp f2 is likely to confer protection against subsequent Aspergillus challenge in CGD mice by activating DCs mediating local (pulmonary) and systemic (splenic) immunity, and increasing the repertoire of antigen-specific type I committed T-cells. HSP110 may also stimulate NK cells to produce IFN-γ. HSP110/Asp f2 may also directly activate innate oxidant-independent host defense pathways in key effector cells, such as neutrophils and macrophages, via TLR 4 activation.

Rationale for Corticosteroid-treated Mice. Several mouse models mimic the iatrogenic immunosuppression observed in the clinic and have been commonly used in studies of experimental aspergillosis. The most common methods include rendering the mouse neutropenic by cyclophosphamide (an alkylating agent) or by antibody depletion, allogeneic bone marrow transplantation, and systemic administration of corticosteroids. All of these approaches have merit, and there is a rationale for evaluating HSP110-based vaccines in each of them. Corticosteroid treatment was selected to specifically evaluate an important principle. High-dose corticosteroids potently inhibit T-cell activation, and may exert a greater suppressive effect on the clonal expansion of naive versus memory T-cells (Brinkmann et al., “Regulation by Corticosteroids of Th1 and Th2 Cytokine Production in Human CD4+ Effector T Cells Generated From CD45RO− and CD45RO+ Subsets,” J Immunol 155:3322-8 (1995), which is hereby incorporated by reference in its entirety). It has been shown that immunization with Aspergillus extract conferred protection against lethal Aspergillus challenge in corticosteroid-treated mice, thus providing a proof of principle that an immunization strategy can be effective in the setting of corticosteroid immunosuppression (Ito et al., “Vaccination of Corticosteroid Immunosuppressed Mice Against Invasive Pulmonary Aspergillosis,” J Infect Dis 186:869-71 (2002), which is hereby incorporated by reference in its entirety). Whether HSP110/Asp f2vaccine remains immunogenic and protective in the setting of corticosteroid immunosuppression will specifically be examined.

Morbidity Experiments. Mice will be sensitized on days 0 and 14 with i.p. HSP110/Asp f2 complex (10 μg Asp f2+25 μg Hsp110 per mouse corresponding to 1:1 molar ratio) and the appropriate controls: vehicle, Asp f2 alone (10 μg/mouse), HSP110 alone (25 μg/mouse), or Asp f2 (10 μg/mouse) together with CpG oligodeoxynucleotide sequences (50 μg/mouse). At 11 days after booster, mice will be challenged intratracheally with an inoculum of Aspergillus conidia known to cause at least 80% mortality by 21 days. In corticosteroid-treated mice, the same hydrocortisone dosing regimen will be used as previously described in experiments involving Aspergillus nidulans (Chang et al., “Virulence of Catalase-Deficient Aspergillus Nidulans in p47(Phox)−/−Mice. Implications for Fungal Pathogenicity and Host Defense in Chronic Granulomatous Disease,” J Clin Invest 101:1843-50 (1998), which is hereby incorporated by reference in its entirety). Systemic hydrocortisone (125 mg/kg s.c.) will be administered daily on days 7 to 11 after the last vaccine booster (or appropriate control) followed by intratracheal Aspergillus challenge. To maintain the state of immunosuppression, corticosteroids will be administered on days 2, 4, 6, and 8 after Aspergillus challenge.

In general, 10 mice per treatment group will be employed to evaluate 21-day survival without morbidity by Kaplan-Meier plots. Mice will be inspected at least twice daily by trained staff and all mice with the following pre-specified signs of morbidity will be sacrificed: inability to feed or drink, labored breathing, listlessness, hunched posture, ruffled fur, or other unanticipated signs of distress. Corticosteroid-treated mice will receive trimethoprim-sulfamethoxazole prophylaxis in the drinking water to prevent bacterial infections. All decisions about euthanasia will be made blinded to the assigned treatment regimen.

The belief that HSP110/Asp f2 sensitization will stimulate innate and Asp f2-specific cellular immunity that will correlate with protection against Aspergillus infection will be tested. Mice will be sacrificed at pre-specified time points to characterize lung histopathology, quantitation of lung fungal burden by PCR, DC activation, Asp f2 specific IgG1 and IgG2a, and Asp f2 specific T-cell responses.

It is believed that the extent of lung fungal burden and disease following Aspergillus challenge will be reduced in mice vaccinated with the HSP110/Asp f2 complex. Ten percent of the LD50 inoculum will be used in experiments that assess fungal burden, histopathology and cytokine responses. Mice (n=5 per treatment group) will be sacrificed on days 7, and 21 after Aspergillus challenge. Quantitative real-time PCR assay using fluorescence resonance energy transfer technology for detection of fungal DNA in lung samples will be performed. Lung disease will be assessed histopathologically and scored in a semi-quantitative fashion (0: no injury; 1: 1-25% involvement; 2: 26-50% involvement; 3: >50% involvement) as previously described (Petraitis et al., “Antifungal Efficacy, Safety, and Single-Dose Pharmacokinetics of LY303366, a Novel Echinocandin B, in Experimental Pulmonary Aspergillosis in Persistently Neutropenic Rabbits,” Antimicrob Agents Chemother 42(11):2898-905 (1998), which is hereby incorporated by reference in its entirety). Necrosis, hyphal invasion, and the predominant inflammatory cell type will be determined. Immunostaining for CD4+ and CD8+ T-cells will be performed on lung sections. Analysis of lung pathology will be done in a blinded fashion with respect to treatment group.

It is believed that sensitization with HSP110/Asp f2 will activate pulmonary and splenic DCs in CGD and corticosteroid mice and that DC activation will correlate with protection against Aspergillus infection. The methods used to assess DC activation in vivo are described above in Example 1. Based on the results in vitro (shown in FIG. 3), it is expected that HSP110 alone and Asp f2 alone will activate DCs in vivo. However, it is expected that the HSP110/Asp f2 complex will be required to stimulate both DC activation and Asp f2-specific type I cellular immunity that will lead to more effective protection against subsequent Aspergillus challenge than sensitization with either HSP110 or Asp f2 alone.

It is expected that HSP110/Asp f2 sensitization will stimulate Asp f2-specific type 1 T-cell responses in immunocompromised mice that will correlate with protection against Aspergillus infection. CGD and corticosteroid-treated mice used to characterize DC activation in vivo (above) will also be used to evaluate Aspergillus antigen-specific T-cell responses. Thoracic lymph nodes and spleens will be harvested 7 and 21 days after Aspergillus challenge (or sham infection with vehicle) and single cell suspensions generated. Lymph node cells and splenocytes (5×10⁵) will be incubated with Asp f2 (10 μg/ml), an irrelevant control recombinant protein (e.g. HER-2/neu, 10 μg/ml), con A (5 μg/ml) or HSP110 (10 μg/ml) in complete medium at 37° C. in an atmosphere of 5% CO₂ for 20-24 h. The proportion of IFN-γ and IL-4 producing cells will be determined by ELISPOT as described above. Once this assay is established using published methods (Lyadova et al., “CD4 T Cells Producing IFN-Gamma in the Lungs of Mice Challenged With Mycobacteria Express a CD27-Negative Phenotype,” Clin Exp Immunol 138(1):21-9 (2004), which is hereby incorporated by reference in its entirety) and it confirms that the yield of T-cells extracted from lungs is adequate, lung ELISPOT assays will be included in the characterization of cellular immunity.

Additional HSP110/Aspergillus antigen complexes will be generated and their immunogenicity will be characterized. Promising candidate vaccines will be defined by their ability to elicit type 1 antigen-specific T-cell responses in vivo as determined by the ELISPOT assay.

A major goal of this supplemental experimental work is to assess relative efficacy for HSP110 complexes as vaccine candidates for fungal infections such as those caused by Aspergillus infection. Each of a number of candidate antigens from a group of over 20 recombinant Aspergillus antigens will be examined (Kurup et al., “Selected Recombinant Aspergillus Fumigatus Allergens Bind Specifically to IgE in ABPA,” Clin Exp Allergy 30:988-93 (2000); Banerjee et al., “Cloning and Expression of Aspergillus Fumigatus Allergen Asp f16 Mediating Both Humoral and Cell-Mediated Immunity in Allergic Bronchopulmonary Aspergillosis (ABPA),” Clin Exp Allergy 31:761-70 (2001), which are hereby incorporated by reference in their entirety). Based on their reported properties, several of these antigens could be justified as a candidate antigen in a vaccine-based strategy of the present invention, however Asp f4 and Asp f16 have been selected for further study. Asp f4 has been shown to be the most potent inducer of proliferation, Th1 differentiation, and expression of activation markers in patients with multiple myeloma (Grazziutti et al., “Recombinant Aspergillus Fumigatus Antigen 4 (Af4) Induces Potent Type 1 Cellular Immune Responses: Implications for Immunotherapy of Aspergillus Infections,” International Society for Cellular Therapy (ISCT) Annual Meeting, Dublin, Ireland May 2004, which his hereby incorporated by reference in its entirety). While immune responses may be different in patients and mice, this encouraging finding in immunocompromised patients provides a rationale for characterizing Asp f4 in the vaccine model of the present invention. Asp f16, a recently characterized Aspergillus antigen in ABPA, has been shown to be immunogenic when paired with CpG sequences and protective against Aspergillus infection in mice (Bozza et al., “Vaccination of Mice Against Invasive Aspergillosis With Recombinant Aspergillus Proteins and CpG Oligodeoxynucleotides as Adjuvants,” Microbes Infect 4:1281-90 (2002), which is hereby incorporated by reference in its entirety).

Candidate vaccines will be prioritized according to their ability to elicit type 1 antigen-specific T-cell responses based on the ELISPOT assay. Type 1 antigen-specific T-cell responses will be defined by the proportion of IFN-γ positive cells and the ratio of IFN-γ/IL-4 producing cells following ex vivo stimulation with the corresponding Aspergillus antigen used for in vivo sensitization.

Rationale and potential limitations of criteria to identify promising vaccine candidates. Since the use of stress protein complexes as a fungal vaccine is a novel concept, evaluation of T-cell phenotypes in response to sensitization is essentially at an exploratory level. There are limited published data to guide these endpoints. It has been shown that the proportion of IFN-γ producing CD4+ T-cells significantly increased in the thoracic lymph nodes and spleens of mice challenged with intratracheal Aspergillus conidia, while IL-4 producing cells were increased after challenge with hyphae (Bozza et al., “Dendritic Cells Transport Conidia and Hyphae of Aspergillus Fumigatus From the Airways to the Draining Lymph Nodes and Initiate Disparate Th Responses to the Fungus,” J Immunol 168: 1362-71 (2002), which is hereby incorporated by reference in its entirety). Because mice were sacrificed 3 days after Aspergillus challenge, the long-term kinetics of T-cell cytokine phenotypes were not evaluated. In a subsequent study using CpG sequences as an adjuvant Asp f16, IFN-γ, IL-4, and IL-10 producing CD4+ lung lymphocytes were assayed 6 days after immunization (Bozza et al., “Vaccination of Mice Against Invasive Aspergillosis With Recombinant Aspergillus Proteins and CpG Oligodeoxynucleotides as Adjuvants,” Microbes Infect 4:1281-90 (2002), which is hereby incorporated by reference in its entirety). The CPG-Asp f16 sensitized group had at least a two-fold greater proportion of IFN-γ positive cells and at least a 50% reduction of IL-4 and IL-10 positive cells compared with sensitization with CpG sequences alone, antigen alone, or untreated. Based in part on this limited database, the following criteria will be used in evaluating promising HSP110-Aspergillus antigen complexes: 1) a significant increase in the proportion of IFN-γ positive CD4+ and/or CD8+ T-cells in HSP110-Aspergillus antigen recipients compared with mice sensitized with Aspergillus antigen alone in response to ex vivo stimulation with the relevant Aspergillus antigen (as determined by the ELISPOT assay); and 2) a significant increase in the ratio of IFN-γ to IL-4 positive cells in HSP110-Aspergillus antigen recipients compared with mice sensitized with Aspergillus antigen alone in response to ex vivo stimulation with the relevant Aspergillus antigen. These endpoints will be used to establish a preliminary rank order of promising candidate vaccines.

While these are useful criteria to exclude vaccine candidates with little to no immunogenicity in vivo, they may not be the optimal predictors of the most effective vaccine candidates. For example, it may be that in mice sensitized with HSP110 complexed with a given Aspergillus antigen, CD4+ and CD8+ T-cells generate a robust IFN-γ response in the absence of ex vivo stimulation that is significantly above the response from mice sensitized with HSP110 or antigen alone. This endpoint may, in turn, be more predictive of the vaccine's ability to confer protection against Aspergillus infection than ex vivo responses to the relevant Aspergillus antigen. Thus, candidate vaccine HSP110 complexes will be evaluated using the pre-specified criteria described above, even through these criteria may need to be modified as additional data are generated.

HSP110 as a vaccine adjuvant has several features that are directly relevant to Aspergillus infection. Whereas most vaccine adjuvants cause humoral responses, HSP110 activates both innate and acquired host defense pathways including: 1) activation of toll like receptors; 2) maturation of DCs; 3) activation of T-cell immunity with polarization to type I cytokines; and 4) IgG subclass switching in favor of IgG2a, a reflection of IFN-γ production. The ability of HSPs to augment multiple pathways involved in the immunologic response to Aspergillus makes this approach highly attractive. Prior sensitization of HSP110/Asp f2 is expected to confer protection both by skewing inflammatory responses to the type I phenotype and by promoting fungal clearance through augmentation of innate and antigen-specific host defense pathways.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A pharmaceutical composition comprising a stress protein complex, wherein the stress protein complex comprises:
a stress protein or polypeptide and
an immunogenic fungal polypeptide.

2. The pharmaceutical composition according to claim 1, wherein the stress protein or polypeptide is covalently complexed with the immunogenic fungal polypeptide.

3. The pharmaceutical composition according to claim 1, wherein the stress protein or polypeptide is non-covalently complexed with the immunogenic fungal polypeptide.

4. The pharmaceutical composition according to claim 1, wherein the stress protein or polypeptide and the immunogenic fungal polypeptide are in the form of a fusion polypeptide.

5. The pharmaceutical composition according to claim 1, wherein the stress protein or polypeptide is an HSP110 or GRP170 polypeptide.

6. The pharmaceutical composition according to claim 5, wherein the stress protein complex further comprises the HSP110 polypeptide further complexed with one or both of HSP70 and HSP25 polypeptides.

7. The pharmaceutical composition according to claim 5, wherein the stress protein complex further comprises a polypeptide selected from the group consisting of members of the HSP70, HSP90, GRP78, and GRP94 stress protein families.

8. The pharmaceutical composition according to claim 1, wherein the immunogenic fungal peptide comprises an Aspergillus antigen.

9. The pharmaceutical composition according to claim 8, wherein the Aspergillus antigen is selected from the group consisting of Asp f1, Asp f2, Asp f3, Asp f4, Asp f6, Asp f7, Asp f9, Asp f13, and Asp f16.

10. The pharmaceutical composition according to claim 1, wherein the complex has been heated to enhance binding of the stress protein or polypeptide to the immunogenic fungal polypeptide.

11. The pharmaceutical composition according to claim 1 further comprising a pharmaceutically acceptable carrier.

12. The pharmaceutical composition according to claim 1 further comprising an adjuvant.

13. A pharmaceutical composition comprising:
a first polynucleotide encoding a stress protein or polypeptide and
a second polynucleotide encoding an immunogenic fungal polypeptide.

14. The pharmaceutical composition according to claim 13, wherein the first polynucleotide is operatively coupled to the second polynucleotide to encode a fusion protein.

15. The pharmaceutical composition according to claim 13, wherein the first polynucleotide encodes an HSP110 or a GRP170 polypeptide.

16. The pharmaceutical composition according to claim 13, wherein the immunogenic fungal peptide comprises an Aspergillus antigen.

17. The pharmaceutical composition according to claim 16, wherein the Aspergillus antigen is selected from the group consisting of Asp f1, Asp f2, Asp f3, Asp f4, Asp f6, Asp f7, Asp f9, Asp f13, and Asp f16.

18. The pharmaceutical composition according to claim 13 further comprising a pharmaceutically acceptable carrier.

19. The pharmaceutical composition according to claim 13 further comprising an adjuvant.

20. A pharmaceutical composition comprising:
an antigen presenting cell modified to present a stress protein or polypeptide and
an immunogenic fungal polypeptide.

21. The pharmaceutical composition according to claim 20, wherein the antigen presenting cell is a dendritic cell or a macrophage.

22. The pharmaceutical composition according to claim 20, wherein the antigen presenting cell is modified by peptide loading.

23. The pharmaceutical composition according to claim 20, wherein the stress protein or polypeptide is an HSP110 or GRP170 polypeptide.

24. The pharmaceutical composition according to claim 23, wherein the HSP110 or GRP170 polypeptide is complexed with the immunogenic fungal polypeptide.

25. The pharmaceutical composition according to claim 24, wherein the HSP110 or GRP170 polypeptide is non-covalently complexed with the immunogenic fungal polypeptide.

26. The pharmaceutical composition according to claim 24, wherein the HSP110 or GRP170 polypeptide is covalently complexed with the immunogenic fungal polypeptide.

27. The pharmaceutical composition according to claim 24, wherein the complex comprises the HSP110 polypeptide further complexed with one or both of HSP70 and HSP25 polypeptides.

28. The pharmaceutical composition according to claim 24, wherein the complex further comprises a polypeptide selected from the group consisting of members of the HSP70, HSP90, GRP78, and GRP94 stress protein families.

29. The pharmaceutical composition according to claim 20, wherein the immunogenic fungal polypeptide comprises an Aspergillus antigen.

30. The pharmaceutical composition according to claim 29, wherein the Aspergillus antigen is selected from the group consisting of Asp f1, Asp f2, Asp f3, Asp f4, Asp f6, Asp f7, Asp f9, Asp f13, and Asp f16.

31. The pharmaceutical composition according to claim 23, wherein the antigen presenting cell is modified by transfection with a first polynucleotide encoding the HSP110 or GRP170 polypeptide and a second polynucleotide encoding the immunogenic fungal polypeptide.

32. The pharmaceutical composition according to claim 28, wherein the first polynucleotide is operatively linked to the second polynucleotide to encode a fusion polypeptide.

33. The pharmaceutical composition according to claim 20 further comprising a pharmaceutically acceptable carrier.

34. The pharmaceutical composition according to claim 20 further comprising an adjuvant.

35. A method of treating or preventing a fungal disease in a subject comprising:
administering to the subject an amount of the pharmaceutical composition according to claim 1 effective to induce in the subject an immune response against the immunogenic fungal polypeptide, whereby the immune response treats or prevents the fungal disease in the subject.

36. The method according to claim 35, wherein the fungal disease is caused by Aspergillus spp.

37. The method according to claim 36, wherein the fungal disease is an infectious disease.

38. The method according to claim 37, wherein the infectious disease is invasive aspergillosis.

39. The method according to claim 36, wherein the fungal disease is an allergic disease.

40. The method according to claim 39, wherein the allergic disease is selected from the group consisting of allergic bronchopulmonary aspergillosis and allergic Aspergillus sinusitis.

41. A method of treating or preventing a fungal disease in a subject comprising:
administering to a subject an amount of the pharmaceutical composition according to claim 13 effective to induce in the subject an immune response against the immunogenic fungal polypeptide, whereby the immune response treats or prevents the fungal disease in the subject.

42. The method according to claim 41, wherein the fungal disease is caused by Aspergillus spp.

43. The method according to claim 42, wherein the disease is an infectious disease.

44. The method according to claim 43, wherein the infectious disease is invasive aspergillosis.

45. The method according to claim 42, wherein the fungal disease is an allergic disease.

46. The method according to claim 45, wherein the allergic disease is selected from the group consisting of allergic bronchopulmonary aspergillosis and allergic Aspergillus sinusitis.

47. A method of treating or preventing a fungal disease in a subject comprising:
administering to a subject an amount of the pharmaceutical composition according to claim 20 effective to induce in the subject an immune response against the immunogenic fungal polypeptide, whereby the immune response treats or prevents the fungal disease in the subject.

48. The method according to claim 47, wherein the fungal disease is caused by Aspergillus spp.

49. The method according to claim 48, wherein the fungal disease is an infectious disease.

50. The method according to claim 49, wherein the infectious disease is invasive aspergillosis.

51. The method according to claim 48, wherein the fungal disease is an allergic disease.

52. The method according to claim 51, wherein the allergic disease is selected from the group consisting of allergic bronchopulmonary aspergillosis and allergic Aspergillus sinusitis.

53. A method of treating a fungal disease in a subject, said method comprising:
activating antigen presenting cells in vitro with a stress protein or polypeptide;
contacting the activated antigen presenting cells with a fungal antigenic peptide; and
introducing the contacted and activated antigen presenting cells into a subject having a fungal disease, thereby treating the fungal disease.

54. The method according to claim 53, wherein the stress protein or polypeptide is an HSP110 polypeptide or a GRP170 polypeptide.

55. The method according to claim 53, wherein the fungal disease is caused by Aspergillus spp.

56. The method according to claim 55, wherein the fungal disease is an infectious disease.

57. The method according to claim 56, wherein the infectious disease is invasive aspergillosis.

58. The method according to claim 55, wherein the fungal disease is an allergic disease.

59. The method according to claim 58, wherein the allergic disease is selected from the group consisting of allergic bronchopulmonary aspergillosis and allergic Aspergillus sinusitis.

60. The method according to claim 53, wherein the antigen presenting cells are dendritic cells or macrophages.

61. A transgenic antigen presenting cell comprising:
a first polynucleotide encoding a stress protein or polypeptide and
a second polynucleotide encoding an immunogenic fungal polypeptide.

62. The transgenic antigen presenting cell according to claim 61, wherein the first polynucleotide is operatively linked to the second polynucleotide to encode a fusion polypeptide.

63. The transgenic antigen presenting cell according to claim 61, wherein the first polynucleotide encodes a heat shock protein or polypeptide or a glucose regulated protein or polypeptide.

64. The transgenic antigen presenting cell according to claim 63, wherein the first polynucleotide encodes a HSP110 polypeptide or a GRP170 polypeptide.

65. The transgenic antigen presenting cell according to claim 61, wherein the second polynucleotide encodes an immunogenic fungal polypeptide comprising an Aspergillus antigen.

66. The transgenic antigen presenting cell according to claim 65, wherein the Aspergillus antigen is selected from the group consisting of Asp f1, Asp f2, Asp f3, Asp f4, Asp f6, Asp f7, Asp f9, Asp f13, and Asp f16.

67. The transgenic antigen presenting cell according to claim 61 further comprising a third polynucleotide encoding a polypeptide selected from the group consisting of members of the HSP70, HSP90, GRP78, and GRP94 stress protein families.

68. The transgenic antigen presenting cell according to claim 61, wherein the antigen presenting cells are dendritic cells or macrophages.
